(19) United States
(12) Patent Application Publication
(10) Pub. No.: US 2024/0351977 A1
(43) Pub. Date: Oct. 24, 2024

(54) Compositions and Methods For Reducing Immune Intolerance and Treating Autoimmune Disorders

(71) Applicant: Lapix Therapeutics, Inc., Boston, MA (US)

(72) Inventors: Anas M. Fathallah, Wakefield, MA (US); Scott D. Larsen, Gobles, MI (US); Abdulraouf Ramadan, Malden, MA (US)

(73) Assignee: Lapix Therapeutics, Inc., Boston, MA (US)

(21) Appl. No.: 18/579,943

(22) PCT Filed: Aug. 12, 2022
(86) PCT No.: PCT/US2022/074903

§ 371 Date: Jan. 17, 2024

Related U.S. Application Data
(60) Provisional application No. 63/233,163, filed on Aug. 13, 2021.

(51) Int. Cl. C07C 237/12; A61K 9/127; A61K 39/00; A61K 39/39; C07C 271/22; C12N 15/86

(52) U.S. Cl. CPC C07C 237/12; A61K 9/127; A61K 39/39; C07C 271/22; C12N 15/86; A61K 2039/542; A61K 2039/55555; C12N 2750/14143

(57) ABSTRACT

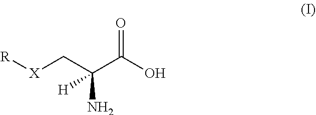

Provided herein are compounds and their pharmaceutically acceptable salts, lipid particles comprising such compounds or pharmaceutically acceptable salts thereof and compositions of the foregoing that can be used to reduce immune intolerance in a subject, for example, to treat autoimmune disorders, or in combination with an antigenic therapy, such as a protein or gene therapy, to improve the efficacy of the antigenic therapy. The compounds have the following structural formula: wherein values for the variables (e.g., X, R) are as described herein.

### RELATED APPLICATION

This application claims the benefit of U.S. Provisional Application No. 63/233,163, filed on Aug. 13, 2021. The entire teachings of this application are incorporated herein by reference.

### BACKGROUND

Enzyme and protein replacement therapy is a successful therapeutic strategy for treating congenital disorders where an endogenous protein is mutated, missing, or otherwise aberrant. However, clinical administration of foreign enzyme or protein is associated with the development of unwanted immune response toward the enzyme or protein. The unwanted immune response could lead to neutralization of the enzyme/protein, or alteration of its pharmacokinetics. In many circumstances, patients do not have alternative therapeutic options, making the unwanted immune response to therapy a major issue facing enzyme and protein replacement therapy recipients.

Similarly, gene therapy offers a promising approach to treat a number of congenital disorders and other diseases. Immunogenicity of the carrier and/or the genetic material carried within is a major challenge to the clinical application of gene therapy. Existing anti-carrier antibodies is a counter-indication to treatment with some approved gene therapies. Furthermore, nascent anti-carrier antibodies can prevent repeat dosing in subjects that receive the first dose of a gene therapy.

Autoimmune disorders are a collection of disorders in which the body lacks or loses tolerance to self-antigens. This results in the body's immune system attacking healthy cells, and can have debilitating and devastating effects. Current approaches to treating autoimmune disorders rely on general immune suppression at the humoral, cellular and/or complement level, rendering patients immunocompromised and susceptible to opportunistic infections.

Accordingly, there is a need for compositions that can reduce immune intolerance to exogenous antigens (e.g., enzyme replacement therapy, gene therapy) or endogenous antigens (e.g., self-antigens causing autoimmune disorders), for example, by mitigating the immunogenicity of enzyme and protein replacement therapy and/or gene therapy, or increasing self-tolerance to self-antigens.

### SUMMARY

The technology described herein relates to tolerance induction for exogenous antigens (e.g., antigen-specific and/or antigen-exclusive tolerance induction), or for self-antigens. The technology is based on engaging and modulating (e.g., activating) the T-cell immunoglobulin mucin protein (TIM) family of receptors.

Provided herein is a compound of the following structural formula:

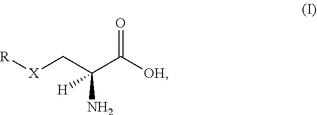

or a pharmaceutically acceptable salt thereof, wherein values for the variables (e.g., R, X) are as described herein.

Also provided herein is a lipid particle comprising one or more lipids, or a pharmaceutically acceptable salt thereof, and a compound of the disclosure.

Also provided herein is a composition (e.g., pharmaceutical composition) comprising a compound of the disclosure.

Also provided herein is a composition (e.g., pharmaceutical composition) comprising a plurality of lipid particles described herein.

Also provided herein are methods of immunotolerizing a subject in need thereof. The methods comprise administering to the subject a therapeutically effective amount of a composition described herein.

Also provided herein are methods of immunotolerizing a subject in need thereof to an antigen and inhibiting or reducing an antigen-specific antibody titer in a subject. The methods comprise administering to the subject the antigen and a therapeutically effective amount of a composition described herein, or administering to the subject a composition described herein comprising the antigen, or an immunogenic fragment of the antigen.

Also provided herein are methods of inducing a population of regulatory T-cells in a subject (e.g., in response to an antigen) and increasing the activity or level of tolerogenic T-cells in a subject. The methods comprise administering to the subject a therapeutically effective amount of a composition described herein (e.g., a composition described herein comprising the antigen, or an immunogenic fragment of the antigen).

Also provided herein are methods of inducing a population of regulatory B-cells in a subject (e.g., in response to an antigen). The methods comprise administering to the subject a therapeutically effective amount of a composition described herein (e.g., a composition described herein comprising the antigen, or an immunogenic fragment of the antigen).

Also provided herein is a method of treating an autoimmune disorder in a subject in need thereof, comprising administering to the subject a therapeutically effective amount of a composition described herein (e.g., a composition described herein comprising self-antigen associated with the autoimmune disorder).

Also provided herein is a method of treating a disease, disorder or condition in a subject in need thereof with an antigenic therapy, comprising administering to the subject the antigenic therapy (e.g., a therapeutically effective amount of the antigenic therapy) and a composition described herein in an amount sufficient to immunotolerize the subject to the antigenic therapy, or a therapeutically effective amount of a composition described herein comprising the antigenic therapy.

Also provided herein is a composition (e.g., pharmaceutical composition) for a use described herein (e.g., treatment of an autoimmune disorder; treatment of a disease, disorder or condition treatable with antigenic therapy), wherein the composition is a composition described herein. Also provided herein is use of a composition described herein for the manufacture of a medicament for a use described herein (e.g., treatment of an autoimmune disorder; treatment of a disease, disorder or condition treatable with antigenic therapy).

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments.

FIG. 1 shows the active ingredient decision tree described in Example 2.

FIG. 2A shows representative images of B-cell clustering within the lymph node. Arrows indicate the cortex of the lymph node.

FIG. 2B shows DilC₁₈ (5) lymphatic uptake and B-cell colocalization 60 minutes after oral gavage.

FIG. 2C shows DMPC lymphatic uptake and B-cell colocalization 60 minutes after oral gavage.

FIG. 2D shows DOPC lymphatic uptake and B-cell colocalization 60 minutes after oral gavage.

FIG. 2E shows DSPC lymphatic uptake and B-cell colocalization 60 minutes after oral gavage.

FIG. 2F shows POPC lymphatic uptake and B-cell colocalization 60 minutes after oral gavage.

FIG. 2G shows percent B-cell/liposome colocalization by lipid type.

FIG. 2H shows representative images of T-cell clustering within the lymph node.

FIG. 2I shows DilC₁₈ (5) lymphatic uptake and T-cell colocalization 60 minutes after oral gavage.

FIG. 2J shows DMPC lymphatic uptake and T-cell colocalization 60 minutes after oral gavage.

FIG. 2K shows DOPC lymphatic uptake and T-cell colocalization 60 minutes after oral gavage.

FIG. 2L shows DSPC lymphatic uptake and T-cell colocalization 60 minutes after oral gavage.

FIG. 2M shows POPC lymphatic uptake and T-cell colocalization 60 minutes after oral gavage.

FIG. 2N shows percent T-cell/liposome colocalization by lipid type.

FIG. 3 shows the changes in percent FoxP3⁺/TIM3⁺CD4 T-cells in response to Compound 1 or Compound 2.

FIG. 4A is a schematic diagram of evaluating the pharmacodynamic (PD) effects of Compound 2 in mice.

FIG. 4B shows the Compound 2 dose-PD relationship in vivo, including the dose-PD model fit and associated confidence interval around the mean model predicted dose-PD. The ED₅₀ and ED₉₀ values are shown.

FIG. 5A is a schematic diagram of a study for determining therapeutic uses of liposomal Compound 2 in the EAE multiple sclerosis (MS) model.

FIG. 5B shows the time to disease onset in the EAE MS model.

FIG. 5C shows the clinical scores of the treated and control groups in the prophylactic EAE MS model.

FIG. 5D shows the clinical scores of the treated and control groups in the therapeutic EAE MS model.

FIG. 5E shows the overall survival of the treated and control groups in the EAE MS model.

FIG. 6 shows representative transmission electron microscopy images of AAV9-CMV^{Chry} encapsulated in DMPC: GL67: Compound 2 (85:5:10) liposomes, where the red arrows and circles indicate encapsulated AAV9-CMV^{Chry}

### DETAILED DESCRIPTION

A description of example embodiments follows.

### Definitions

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

When introducing elements disclosed herein, the articles “a,” “an,” “the,” and “said” are intended to mean that there are one or more of the elements. Further, the one or more elements may be the same or different.

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., ±10%, ±5% or ±1% of a given value. It is to be understood that the term “about” can precede any particular value specified herein, except for particular values used in the Exemplification.

“Alkyl” refers to a branched or straight-chain, monovalent, hydrocarbon radical having the specified number of carbon atoms. Thus, “(C₁-C₈)alkyl” refers to a radical having from 1-8 carbon atoms in a branched or linear arrangement. In some aspects, alkyl is (C₁-C₃₀)alkyl, e.g., (C₅-C₃₀)alkyl, (C₁-C₂₅)alkyl, (C₅-C₂₅)alkyl, (C₁₀-C₂₅)alkyl, (C₁₅-C₂₅)alkyl, (C₁₀-C₂₀)alkyl, (C₁₅-C₂₀)alkyl, (C₁-C₁₅)alkyl, (C₁-C₁₀)alkyl, (C₁-C₆)alkyl, or (C₁-C₅)alkyl. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like. In some aspects, alkyl is optionally substituted, e.g., with one or more substituents described herein.

“Alkenyl” refers to a branched or straight-chain, monovalent, hydrocarbon radical having at least one carbon-carbon double bond and the specified number of carbon atoms. Thus, “(C₂-C₈)alkenyl” refers to a radical having at least one carbon-carbon double bond and from 2-8 carbon atoms in a branched or linear arrangement. In some aspects, alkenyl is (C₁-C₃₀)alkenyl, e.g., (C₅-C₃₀)alkenyl, (C₁-C₂₅)alkenyl, (C₅-C₂₅)alkenyl, (C₁₀-C₂₅)alkenyl, (C₁₅-C₂₅)alkenyl, (C₁₀-C₂₀)alkenyl, (C₁₅-C₂₀)alkenyl, (C₁-C₁₅)alkenyl, (C₁-C₁₀)alkenyl, (C₁-C₆)alkenyl, or (C₁-C₅)alkenyl. Examples of alkenyl groups include ethenyl, 2-propenyl, 1-propenyl, 2-methyl-1-propenyl, 1-butenyl, 2-butenyl, 1-pentenyl, 2-pentenyl, 3-pentenyl, allyl, 1, 3-butadienyl, 1,3-dipentenyl, 1,4-dipentenyl, 1-hexenyl, 1,3-hexenyl, 1,4-hexenyl, 1,3,5-trihexenyl, 2,4-dihexenyl, and the like. In some aspects, alkenyl is optionally substituted, e.g., with one or more substituents described herein.

“Aryl” refers to a monocyclic or polycyclic (e.g., bicyclic, tricyclic), aromatic, hydrocarbon ring system having the specified number of ring atoms, and includes aromatic rings fused to non-aromatic rings, as long as one of the fused rings is an aromatic hydrocarbon. Thus, “(C₆-C₁₅) aryl” refers to a ring system having from 6-15 ring atoms. Examples of aryl include phenyl, naphthyl and fluorenyl. In some aspects, aryl is optionally substituted, e.g., with one or more substituents described herein.

“Heteroaryl” refers to a monocyclic or polycyclic (e.g., bicyclic, tricyclic), aromatic, hydrocarbon ring system having the specified number of ring atoms, wherein at least one carbon atom in the ring system has been replaced with a heteroatom selected from nitrogen, sulfur and oxygen. Thus, “(C₅-C₁₅) heteroaryl” refers to a heteroaromatic ring system having from 5-15 ring atoms consisting of carbon, nitrogen, sulfur and oxygen. “Heteroaryl” includes heteroaromatic rings fused to non-aromatic rings, as long as one of the fused rings is a heteroaromatic hydrocarbon. A heteroaryl can contain 1, 2, 3 or 4 (e.g., 1, 2 or 3) heteroatoms independently selected from nitrogen, sulfur and oxygen. Typically, heteroaryl is (C₅-C₂₀) heteroaryl, e.g., (C₅-C₁₅) heteroaryl, (C₅-C₁₂) heteroaryl, C₅ heteroaryl or C₆ heteroaryl. Monocyclic heteroaryls include, but are not limited to, furan, oxazole, thiophene, triazole, triazene, thiadiazole, oxadiazole, imidazole, isothiazole, isoxazole, pyrazole, pyridazine, pyridine, pyrazine, pyrimidine, pyrrole, tetrazole and thiazole. Bicyclic heteroaryls include, but are not limited to, indolizine, indole, isoindole, indazole, benzimidazole, benzofuran, benzothiazole, purine, quinoline, isoquinoline, cinnoline, phthalazine, quinazoline, quinoxaline, naphthyridine and pteridine. In some aspects, heteroaryl is optionally substituted, e.g., with one or more substituents described herein.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Halogen” and “halo” are used interchangeably herein and each refers to fluorine, chlorine, bromine, or iodine. In some aspects, halo is fluoro, chloro or bromo. In some aspects, halo is fluoro.

“Haloalkyl” includes mono, poly, and perhaloalkyl groups, wherein each halogen is independently selected from fluorine, chlorine, bromine and iodine (e.g., fluorine, chlorine and bromine), and alkyl is as described herein. In one aspect, haloalkyl is perhaloalkyl (e.g., perfluoroalkyl). Examples of haloalkyl include, but are not limited to, trifluoromethyl and pentafluoroethyl.

“Haloalkoxy” refers to a haloalkyl radical attached through an oxygen linking atom, wherein haloalkyl is as described herein. Examples of haloalkoxy include, but are not limited to, trifluoromethoxy.

The term “substituted” refers to replacement of a hydrogen atom with a suitable substituent. Typically, the suitable substituent replaces a hydrogen atom bound to a carbon atom, but a substituent may also replace a hydrogen bound to a heteroatom, such as a nitrogen, oxygen or sulfur atom. It will be understood that “substitution” or “substituted with” includes the implicit proviso that such substitution is in accordance with permitted valence of the substituted atom. It is also preferred that the substituent, and the substitution, result in a stable compound, e.g., which does not spontaneously undergo transformation such as by rearrangement, cyclization, elimination, etc. Suitable substituents for use herein include acyclic and cyclic, branched and unbranched, carbocyclic and heterocyclic, aromatic and non-aromatic substituents of organic compounds. For example, suitable substituents can include halogen, hydroxyl, carbonyl (such as carboxyl, alkoxycarbonyl, formyl, or acyl), thiocarbonyl (such as thioester, thioacetate, or thioformate), alkyl, alkoxy, alkylthio, acyloxy, phosphoryl, phosphate, phosphonate, amino, amido, amidine, imine, cyano, nitro, azido, sulfhydryl, alkylthio, sulfate, sulfonate, sulfamoyl, sulfonamido, sulfonyl, cycloalkyl, heterocyclyl, aralkyl, aryl or heteroaryl. It will be understood by those skilled in the art that substituents can themselves be substituted, if appropriate. Accordingly, substituents can further include an acetamide, for example.

The permissible substituents can be one or more and the same or different for appropriate organic compounds. Thus, an “optionally substituted” group is, in some aspects, substituted with 0-5 (e.g., 0-3, 0, 1, 2, 3, 4, 5) substituents independently selected from halo, (C₁-C₆)alkoxy, (C₁-C₆)haloalkoxy, (C₁-C₆)alkyl or (C₁-C₆)haloalkyl, or optionally substituted (C₆-C₁₅) aryl or (C₅-C₁₅) heteroaryl. In some aspects, an optionally substituted aryl or heteroaryl is substituted with 0-5 (e.g., 0-3, 0, 1, 2, 3, 4, 5) substituents independently selected from halo, (C₁-C₆)alkoxy, (C₁-C₆)haloalkoxy, (C₁-C₆)alkyl or (C₁-C₆)haloalkyl. In some aspects, an optionally substituted” aryl or heteroaryl is substituted with 0-5 (e.g., 0-3, 0, 1, 2, 3, 4, 5) substituents independently selected from halo, (C₁-C₃)alkoxy, (C₁-C₃)haloalkoxy, (C₁-C₃)alkyl or (C₁-C₃)haloalkyl. In some aspects, an optionally substituted alkyl or alkenyl is substituted with 0-5 (e.g., 0-3, 0, 1, 2, 3, 4, 5) substituents independently selected from halo (e.g., fluoro), (C₁-C₆)alkoxy, (C₁-C₆)haloalkoxy (e.g., (C₁-C₆) fluoroalkoxy), (C₆-C₁₅) aryl or (C₅-C₁₅) heteroaryl.

The term “optionally substituted”, as used herein, means that substitution is optional and, therefore, it is possible for the atom or moiety designated as “optionally substituted” to be unsubstituted or substituted. In some aspects, an optionally substituted group is unsubstituted. In some aspects, an optionally substituted group is substituted. Unless otherwise indicated, e.g., as with the terms “substituted” or “optionally substituted,” a group designated herein is unsubstituted.

As used herein, the term “compound of the disclosure” refers to a compound of any of the structural formulas depicted herein (e.g., a compound of Structural Formula I, an exemplified compound), as well as isomers, such as stereoisomers (including diastereoisomers, enantiomers and racemates) and tautomers, thereof, isotopically labeled variants thereof (including those with deuterium substitutions), and inherently formed moieties (e.g., polymorphs and/or solvates, such as hydrates) thereof. When a moiety is present that is capable of forming a salt, then salts are included as well, in particular, pharmaceutically acceptable salts thereof.

Compounds of the disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers (including cis and trans double bond isomers), conformational isomers (including rotamers and atropisomers), tautomers) and intermediate mixtures, with all possible isomers and mixtures thereof being included, unless otherwise indicated.

When a disclosed compound is depicted by structure without indicating the stereochemistry, and the compound has one or more chiral centers, it is to be understood that the structure encompasses one enantiomer or diastereomer of the compound separated or substantially separated from the corresponding optical isomer(s), a racemic mixture of the compound and mixtures enriched in one enantiomer or diastereomer relative to its corresponding optical isomer(s). When a disclosed compound is depicted by a structure indicating stereochemistry, and the compound has one or more chiral centers, the stereochemistry indicates absolute configuration of the substituents around the one or more chiral centers. “R” and “S” can also or alternatively be used to indicate the absolute configuration of substituents around one or more chiral carbon atoms. D− and L− can also or alternatively be used to designate stereochemistry.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$ee = \vert \frac{R - S}{R + S} \vert \times 100 \% ,$$

where R and S represent the respective fractions of each enantiomer in a mixture, such that R+S=1. An enantiomer may be present in an ee of at least or about 50%, about 60%, about 70%, about 80%, about 90%, about 95%, about 98%, about 99% or about 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D 4 … )}{D 1 + ( D 2 + D 3 + D 4 … )} \vert \times 100 \% ,$$

where D1 and (D2+D3+D4 . . . ) represent the respective fractions of each diastereomer in a mixture, such that D1+ (D2+D3+D4 . . . )=1. A diastereomer may be present in a de of at least or about 50%, about 60%, about 70%, about 80%, about 90%, about 95%, about 98%, about 99% or about 99.9%.

Unless otherwise stated, compounds of the disclosure include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. In all provided structures, any hydrogen atom can also be independently selected from deuterium (²H), tritium (³H) and/or fluorine (¹⁸F). Such compounds are useful, for example, as analytical tools, as probes in biological assays, or as therapeutic agents in accordance with the present invention.

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as “solvates” or “hydrates.” A “hydrate” is a compound that exists in a composition with one or more water molecules. A hydrate can include water in stoichiometric quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Antigen,” as used herein, refers to any substance that can be recognized by the immune system. “Antigen” broadly encompasses proteins, such as enzymes, peptides, such as polypeptides, carbohydrates, such as polysaccharides, haptens, nucleic acids and grafts. An antigen can be a self-antigen, an antigen produced, under normal conditions or as part of a disorder, by the body, or a foreign antigen, a non-self-antigen. Examples of self-antigens include self-antigens associated with autoimmune disorders, including any of the self-antigens described herein. Examples of foreign antigens include antigenic therapies (e.g., therapeutic proteins, gene therapies, cellular therapies), allergens and alloantigens.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“A therapeutically effective amount” is an amount effective, at dosages and for periods of time necessary, to achieve a desired therapeutic result (e.g., induction of immune tolerance, treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some aspects, a subject is a human.

### Compounds

A first embodiment provides a compound of the following structural formula:

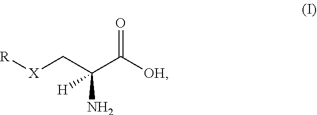

or a pharmaceutically acceptable salt thereof, wherein:
X is —N(R¹)C(O)—, —N(R¹)C(O)O—, —N(R¹)C(O)N(R²)—, —N(R¹)—, —N(R¹) SO₂—, —O—, —S—, —S(O)—, —S(O)₂— or —OP(O)₂O—;
R is (C₁-C₃₀)alkyl or (C₁-C₃₀)alkenyl optionally substituted with one or more fluoro;
R¹ is H or (C₁-C₅)alkyl; and
R² is H or (C₁-C₅)alkyl.

In a first aspect of the first embodiment, X is —N(R¹)C(O)— or —N(R¹)C(O)O—. Values for the remaining variables are as described in the first embodiment.

In a second aspect of the first embodiment, X is —N(R¹)C(O)—* or —N(R¹)C(O)O—*, wherein * indicates the point of attachment of X to R. Values for the remaining variables are as described in the first embodiment, or first aspect thereof.

In a third aspect of the first embodiment, X is —N(R¹)C(O)—*. Values for the remaining variables are as described in the first embodiment, or first or second aspect thereof.

In a fourth aspect of the first embodiment, X is —N(R¹)C(O)O—*. Values for the remaining variables are as described in the first embodiment, or first through third aspects thereof.

In a fifth aspect of the first embodiment, R is (C₅-C₃₀)alkyl or (C₅-C₃₀)alkenyl optionally substituted with one or more fluoro. Values for the remaining variables are as described in the first embodiment, or first through fourth aspects thereof.

In a sixth aspect of the first embodiment, R is (C₅-C₃₀)alkyl optionally substituted with one or more fluoro. Values for the remaining variables are as described in the first embodiment, or first through fifth aspects thereof.

In a seventh aspect of the first embodiment, R¹ and R² are each H. Values for the remaining variables are as described in the first embodiment, or first through sixth aspects thereof.

In an eighth aspect of the first embodiment, X is —N(R¹)C(O)—*, —N(R¹)C(O)O—*, —N(R¹)C(O) N(R²)—*, —N(R¹)—, —N(R¹) SO₂—*, —O—, —S—, —S(O)—, —S(O)₂— or —OP(O)₂O—, wherein * indicates the point of attachment of X to R. Values for the remaining variables are as described in the first embodiment, or first through seventh aspects thereof.

In a ninth aspect of the first embodiment, R is (C₁₀-C₂₅)alkyl or (C₁₀-C₂₅)alkenyl optionally substituted with one or more fluoro. Values for the remaining variables are as described in the first embodiment, or first through eighth aspects thereof.

In a tenth aspect of the first embodiment, R is (C₁₀-C₂₀)alkyl or (C₁₀-C₂₀)alkenyl optionally substituted with one or more fluoro. Values for the remaining variables are as described in the first embodiment, or first through ninth aspects thereof.

In an eleventh aspect of the first embodiment, R is (C₁₅-C₂₅)alkyl or (C₁₅-C₂₅)alkenyl optionally substituted with one or more fluoro. Values for the remaining variables are as described in the first embodiment, or first through tenth aspects thereof.

In a twelfth aspect of the first embodiment, R is (C₁₅-C₂₀)alkyl or (C₁₅-C₂₀)alkenyl optionally substituted with one or more fluoro. Values for the remaining variables are as described in the first embodiment, or first through eleventh aspects thereof.

A second embodiment provides a compound of the following structural formula:

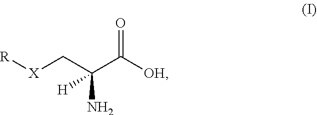

or a pharmaceutically acceptable salt thereof, wherein:
X is —N(R¹)C(O)—* or —N(R¹)C(O)O—*, wherein * indicates the point of attachment of X to R;
R is (C₅-C₃₀)alkyl or (C₅-C₃₀)alkenyl optionally substituted with one or more fluoro; and R¹ is H or (C₁-C₅)alkyl,
provided the compound is not(S)-2-amino-3-((2E,4E)-hexa-2,4-dienamido) propanoic acid, (S)-2-amino-3-hexanamidopropanoic acid, (S)-2-amino-3-heptanamidopropanoic acid, (S)-2-amino-3-octanamidopropanoic acid or(S)-2-amino-3-palmitamidopropanoic acid, or a salt of any of the foregoing.

Alternative values for the variables are as described in the first embodiment, or any aspect thereof.

In a first aspect of the second embodiment, R¹ is H. Values for the remaining variables are as described in the first embodiment, or any aspect thereof, or the second embodiment.

Examples of compounds of Structural Formula I include:

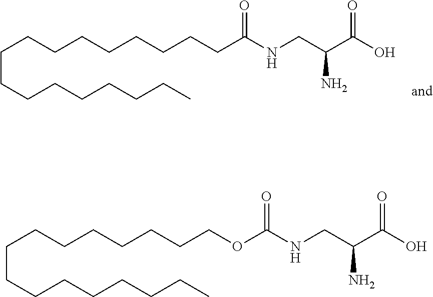 [SMILES: CCCCCCCCCCCCCCCCCC(=O)NC[C@H](N)C(=O)O ; CCCCCCCCCCCCCCCCOC(=O)NC[C@H](N)C(=O)O]

or a pharmaceutically acceptable salt of the foregoing.

Methods of making compounds of Structural Formula I are described herein in the Exemplification.

### Compositions and Kits

Typically, for administration to a subject, a compound of the disclosure is formulated with one or more pharmaceutically acceptable carriers. The disclosure provides such compositions, including pharmaceutical compositions. Thus, one embodiment is a composition (e.g., pharmaceutical composition) comprising a compound of the disclosure and a pharmaceutically acceptable carrier. The compositions described herein can be used in the methods described herein, e.g., to supply a compound of the disclosure.

Compounds and compositions described herein can also be in the form of formulations of lipid particles, such as liposomal formulations. Thus, one embodiment is a lipid particle (e.g., a liposome) comprising one or more lipids and a compound of the disclosure.

Also provided herein is a solid lipid particle (e.g., liposome) comprising at least one phospholipid (e.g., a phospholipid containing a C₄-C₃₀ acyl chain, such as a saturated C₄-C₃₀ acyl chain, as in dimyristoylphosphatidylcholine (DMPC)) and a therapeutic agent (e.g., a compound of the disclosure) that can embed in a lipid bilayer of the lipid particle. It has been found that oral administration of such solid lipid particles can be used to target the lipid particle (and thereby the therapeutic agent) to immune cells and/or lymph node(s), for example, and thereby enhance colocalization of the lipid particles and immune cells (e.g., in the lymph nodes) and/or enhance lymph node uptake of the lipid particles.

As used herein, “lipid particle” refers to a particle comprising at least one lipid, e.g., a phospholipid, such as a lysophospholipid. Examples of lipid particles include, liposomes, micelles and lipid nanoparticles. Lipid particles, such as liposomes, can be unilamellar or multilamellar. Lipid particles, such as liposomes, can have fluidic lipid membranes, or gel-like or solid lipid membranes, for example, lipid membranes that melt above normal body temperature of a human, or about 37° C. In some aspects, a lipid particle is a liposome. In some aspects, a lipid particle is a lipid nanoparticle. In some aspects, a lipid particle is solid. In some aspects, a lipid particle has a melting temperature above about 37° C., e.g., above about 40° C., above about 45° C., above about 50° C., above about 55° C. or about 55° C.

Examples of phospholipids include dimyristoylphosphatidylcholine (DMPC), 1,2-dioleoyl-sn-glycero-3-phosphocholine 18:1 Δ9-Cis PC (DOPC), 1,2-distearoyl-sn-glycero-3-phosphocholine 18:0 (DSPC), 1-palmitoyl-2-oleoyl-glycero-3-phosphocholine 16:0-18:1 (POPC), phosphatidylserine (PS), phosphatidylcholine (PC), phosphatidylethanolamine, phosphatidyl inositol, bisphosphatidyl glycerol, phosphatidic acid, phosphatidyl alcohol and phosphatidyl glycerol. Phospholipids can be saturated or unsaturated, i.e., contain one or more units of unsaturation, and can contain acyl chains of a variety of lengths. In some aspects, a phospholipid contains a C₄-C₃₀ acyl chain, e.g., a C₈-C₂₆, C₁₂-C₂₂, C₁₀-C₂₅, C₁₄-C₁₈ or C₁₆-C₂₆ acyl chain. Phospholipids can be obtained from various sources, both natural and synthetic. For example, PS can be obtained from porcine brain PS or plant-based soy (soya bean) PS. Egg PC and PS and synthetic PC are available commercially. In some aspects, a phospholipid is not PS, or a salt thereof (e.g., pharmaceutically acceptable salt thereof).

Other lipids suitable for inclusion in the lipid particles described herein include N⁴-cholesteryl-spermine, or a salt thereof, such as N⁴-cholesteryl-spermine HCl salt. N⁴-cholesteryl-spermine HCl salt is also known as Genzyme Lipid 67 (GL67), and is a cholesterol derivatized with spermine to create a cationic lipid HCl salt.

Typically, the molar percentage of a therapeutic agent (e.g., compound of the disclosure) in a lipid particle (e.g., liposome) comprising the therapeutic agent will be from about 1% to about 50%, e.g., from about 1% to about 35%, from about 1% to about 25%, from about 1% to about 15%, from about 3% to about 10%, from about 5% to about 50%, from about 5% to about 45%, from about 15% to about 40%, from about 25% to about 35%, about 1%, about 2%, about 3%, about 4%, about 5%, about 6%, about 7%, about 8%, about 9%, about 10%, about 15%, about 25%, about 30% or about 35%. In some aspects, the molar percentage of a therapeutic agent (e.g., compound of the disclosure) in a lipid particle (e.g., liposome) comprising the therapeutic agent will be less than 35%, e.g., less than 30%, less than 15%, or from about 1% to about 10%.

Typically, the molar percentage of lipid (taken individually or collectively) in a lipid particle (e.g., liposome) described herein will be from about 50% to about 99%, e.g., from about 50% to about 75%, from about 85% to about 99%, about 70%, about 75%, about 85%, about 90%, about 91%, about 92%, about 93%, about 94%, about 95%, about 96%, about 97%, about 98% or about 99%. The molar percentage of each lipid in a lipid particle (e.g., liposome) described herein can be from about 1% to about 99%, e.g., from about 1% to about 50%, from about 1% to about 35%, from about 1% to about 25%, from about 1% to about 15%, from about 3% to about 10%, from about 5% to about 50%, from about 5% to about 45%, from about 15% to about 40%, from about 25% to about 35%, about 1%, about 2%, about 3%, about 4%, about 5%, about 6%, about 7%, about 8%, about 9%, about 10%, about 15%, about 25%, about 30% or about 35%.

A compound of the disclosure can be encapsulated within a lipid particle, such as a liposome, described herein, bound (covalently or non-covalently) to a lipid head group or, preferably, embedded, in whole or in part, covalently or non-covalently, in a lipid bilayer (e.g., of a liposome). Without wishing to be bound by any particular theory, it is believed that compounds of the disclosure may embed in a lipid bilayer of a liposome so as to leave the amino acid residue of the compound of the disclosure exposed to the exterior of the liposome, thereby mimicking the natural surface presentation of, for example, PS.

In some aspects, the one or more lipids comprises a phospholipid, or a pharmaceutically acceptable salt thereof, e.g., 1,2-dimyristoyl-sn-glycero-3-phosphocholine (DMPC), or a pharmaceutically acceptable salt thereof. In some aspects, the phospholipid is a saturated phospholipid, e.g., a saturated phospholipid containing a C₄-C₃₀ acyl chain. In some aspects, the phospholipid is unsaturated, e.g., an unsaturated phospholipid containing a C₄-C₃₀ acyl chain. In some aspects, the phospholipid is selected from DMPC, DSPC, DOPC or POPC, or a pharmaceutically acceptable salt of the foregoing. In some aspects, the phospholipid is DMPC or DSPC, or a pharmaceutically acceptable salt of the foregoing.

In some aspects, a lipid particle (e.g., liposome) further comprises an antigen, such as any of the antigens described herein. Thus, in some aspects, a lipid particle further comprises a gene therapy. In some further aspects, the gene therapy comprises DNA and/or RNA and a viral vector. In some aspects, the viral vector is derived from an adeno-associated virus (AAV), such as a recombinant AAV. In some aspects, the AAV is AAV9. Other examples of viral vectors suitable for use in the context of the present disclosure include viral vectors derived from retrovirus, herpes virus, adenovirus, lentivirus, rabies virus, lentivirus, VSV, poxvirus (e.g., vaccinia virus, variola virus, canarypox), reovirus, semliki forest virus, yellow fever virus, sindbis virus, togavirus, baculovirus, bacteriophages, alphavirus, and flavavirus. In some aspects, the antigen, e.g., gene therapy comprising DNA and/or RNA and a viral vector, is encapsulated within the lipid particle.

Lipid particles further comprising an antigen, and formulations comprising such lipid particles, are expected to be particularly useful for applications involving delivery of a gene therapy (e.g., a gene therapy comprising DNA and/or RNA) to a subject. The lipid particles are expected to promote co-presentation of the gene therapy and the compound of the disclosure to the immune system. Such particles can be formulated for oral and/or parenteral (e.g., subcutaneous, intramuscular, intravenous, intradermal) administration, e.g., as by injection.

Another embodiment is a composition (e.g., pharmaceutical composition) comprising a plurality of lipid particles (e.g., a plurality of lipid particles comprising a compound of the disclosure). In some aspects, a composition further comprises a pharmaceutically acceptable carrier.

Compositions described herein and, hence, compounds of the disclosure, may be administered orally, parenterally (including subcutaneously, intramuscularly, intravenously and intradermally), by inhalation spray, topically, rectally, nasally, buccally, vaginally or via an implanted reservoir. The terms “parenteral” and “parenterally,” as used herein, include subcutaneous, intracutaneous, intravenous, intramuscular, intraocular, intravitreal, intra-articular, intra-arterial, intra-synovial, intrasternal, intrathecal, intralesional, intrahepatic, intraperitoneal, intralesional and intracranial injection or infusion techniques. In some aspects, a composition described herein is administrable intravenously and/or intraperitoneally. In some aspects, a composition described herein is administrable orally. In some aspects, a composition described herein is administrable subcutaneously. Preferably, a composition described herein is administered orally, subcutaneously, intraperitoneally or intravenously.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the compound of the disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

A compound of the disclosure can also be in micro-encapsulated form with one or more excipients, as noted above. In such solid dosage forms, the compound can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another aspect, a compound of the disclosure can be provided in an extended (or “delayed” or “sustained”) release composition. This delayed-release composition comprises the compound of the disclosure and a delayed-release component. Such a composition allows targeted release of the compound, for example, into the lower gastrointestinal tract, for example, into the small intestine, the large intestine, the colon and/or the rectum. In certain aspects, a delayed-release composition further comprises an enteric or pH-dependent coating, such as cellulose acetate phthalates and other phthalates (e.g., polyvinyl acetate phthalate, methacrylates (Eudragits)). Alternatively, the delayed-release composition can provide controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

Compositions described herein can also be administered subcutaneously, intraperitoneally or intravenously, e.g., in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, dextrose, water, Ringer's solution, lactated Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

Compositions described herein can also be administered in the form of suppositories for rectal administration. These can be prepared by mixing a compound of the disclosure with a suitable non-irritating excipient that is solid at room temperature but liquid at rectal temperature and, therefore, will melt in the rectum to release the drug. Such materials include cocoa butter, beeswax and polyethylene glycols.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For other topical applications, the compositions can be formulated in a suitable ointment containing the active component suspended or dissolved in one or more carriers. Carriers for topical administration of a compound described herein include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the active compound suspended or dissolved in one or more pharmaceutically acceptable carriers. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. Suitable carriers also include, but are not limited to, mineral oil, sorbitan monostearate, polysorbate 60, cetyl esters wax, cetearyl alcohol, 2-octyldodecanol, benzyl alcohol and water and penetration enhancers.

For ophthalmic use, compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents. Without wishing to be bound by any particular theory, it is believed that local delivery of a composition described herein, as can be achieved by nasal aerosol or inhalation, for example, can reduce the risk of systemic consequences of the composition, for example, consequences for red blood cells.

Other pharmaceutically acceptable carriers that can be used in the compositions described herein include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives thereof, such as hydroxyalkylcyclodextrins, including hydroxylpropyl-β-cyclodextrins, such as 2- and/or 3-hydroxypropyl-β-cyclodextrins, or other solubilized derivatives thereof can also be advantageously used as a pharmaceutically acceptable carrier in the compositions described herein, e.g., to enhance delivery of agents described herein.

One embodiment is a composition comprising a compound of the disclosure (e.g., a plurality of lipid particles comprising a compound of the disclosure, such as any of the lipid particles described herein) and a cyclodextrin or chemically modified derivative thereof. In some aspects, the cyclodextrin or chemically modified derivative thereof comprises a hydroxyalkylcylclodextrin, e.g., a hydroxypropyl-β-cyclodextrin. In some aspects, the compound of the disclosure and the cyclodextrin (e.g., hydroxyalkylcylclodextrin, such as hydroxypropyl-β-cyclodextrin) are present in a ratio of from about 1 to about 50 weight/weight (w/w) to about 1 to about 250 w/w, e.g., from about 1 to about 50 w/w to about 1 to about 100 w/w, about 1 to about 80 w/w or about 1 to about 166 w/w. In some aspects, the composition further comprises a diluent, such as water. In some aspects, the composition further comprises a sweetening agent and/or flavoring agent.

In some aspects, the composition is a liquid dosage form; in further aspects, the composition is a liquid dosage form for oral administration.

In some aspects, a composition described herein further includes one or more additional therapeutic agents, e.g., for use in combination with a compound of the disclosure.

Some embodiments provide a combination (e.g., pharmaceutical combination) comprising a compound of the disclosure (e.g., a composition described herein comprising a compound of the disclosure) and one or more additional therapeutic agents (e.g., one or more compositions comprising one or more additional therapeutic agents). Such combinations are particularly useful as, for example, when the compound of the disclosure and the one or more additional therapeutic agents are to be administered separately. In a combination provided herein, the compound of the disclosure and the one or more additional therapeutic agents can be administrable by the same route of administration or by different routes of administration.

One embodiment is a kit comprising a compound of the disclosure (e.g., a composition described herein comprising a compound of the disclosure) and an antigen (e.g., any of the antigens described herein, such as an antigenic therapy). In one aspect, the kit comprises a therapeutically effective amount of the compound of the disclosure (e.g., an amount sufficient to immunotolerize a subject to an antigen with which it is intended to be administered; a therapeutically effective amount of the compound to treat a disease, disorder or condition described herein). In some aspects, wherein the antigen is an antigenic therapy, the kit comprises a therapeutically effective amount of the antigenic therapy to treat the disease, disorder or condition. In some aspects, a kit further comprises an additional therapeutic agent(s) (e.g., a composition comprising an additional therapeutic agent(s)). In some aspects, the kit further comprises written instructions for administering the compound of the disclosure and/or the antigen and/or the additional agent(s) to a subject to treat a disease, disorder or condition described herein.

Suitable additional therapeutic agents include those described herein with respect to combination therapies.

The compositions described herein can be provided in unit dosage form. The amount of active ingredient that can be combined with a carrier to produce a unit dosage form will vary depending, for example, upon the subject being treated and the particular mode of administration. Typically, a unit dosage form will contain from about 1 to about 1,000 mg of active ingredient(s), e.g., from about 1 to about 500 mg, from about 1 to about 250 mg, from about 1 to about 150 mg, from about 0.5 to about 100 mg, or from about 1 to about 50 mg of active ingredient(s). In some aspects, a unit dosage form contains from about 0.01 mg to about 100 mg of active ingredient(s), e.g., from about 0.1 mg to about 50 mg, from about 0.1 mg to about 10 mg, from about 0.5 mg to about 50 mg of active ingredient(s). In some aspects, a unit dosage form contains from about 1 mg to about 5,000 mg of active ingredient(s) e.g., from about 10 mg to about 2,500 mg, from about 15 mg to about 1,000 mg or from about 100 mg to about 1,000 mg of active ingredient(s). In some aspects, a unit dosage form contains about 15 mg, about 30 mg, about 50 mg, about 100 mg, about 125 mg or about 150 mg of active ingredient(s).

In some aspects, the concentration of one or more therapeutic agents provided in a pharmaceutical composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v; and/or greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some aspects, the concentration of one or more therapeutic agents provided in a pharmaceutical composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v. In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Methods of Use

It has now been found that various compounds of the present disclosure and compositions described herein are capable of binding TIM, e.g., with higher affinity than its natural ligand, phosphatidyl serine (PS), and diminishing the immune response.

One embodiment is a method of modulating the expression or activity of a T cell immunoglobulin and mucin domain (TIM) receptor, comprising contacting a cell (e.g., a cell expressing a TIM receptor, such as an immune cell) with a compound of the disclosure (e.g., a therapeutically effective amount of a compound of the disclosure). TIM receptors are type 1 cell-surface glycoproteins, and TIM1, TIM3 and TIM4, TIM receptors expressed in humans, have been identified as phosphatidylserine receptors. TIM1 is preferentially expressed on T-helper 2 cells, and operates as a potent costimulatory molecule for T-cell activation. TIM3 is preferentially expressed on T-helper 1 cells, type 1 T-cells and dendritic cells, and generates an inhibitory signal resulting in apoptosis of T-helper 1 cells and type 1 T-cells. TIM4 is expressed on antigen-presenting cells, and mediates phagocytosis of apoptotic cells, thereby promoting tolerance. In some aspects, the TIM receptor is a TIM3 receptor. In some aspects, the TIM receptor is a TIM4 receptor. In some aspects, the TIM receptor is a TIM1 receptor. “TIM” is also referred to, for example, in the literature, as “Tim.”

It has now also been found that agonists of TIM receptors inhibit the activity of at least toll-like receptor (TLR) 3 and TLR7, without substantially inhibiting the activity of TLRs 2 and 4, which largely recognize patterns presented by bacteria. Toll-like receptors (TLRs) form a family of pattern recognition receptors that are expressed on innate immune cells, and constitute the immune system's first line of defense against microbes. To date, ten human subtypes of TLRs have been identified. TLRs 1, 2, 4, 5, 6 and 10 are expressed on the cell surface, and TLRs 3, 7, 8 and 9 are localized to the endoplasmic reticulum, endosomes and lysosomes. TLRs 1, 2 and 6 recognize and bind to bacterial lipoproteins and glycolipids. TLRs 3, 7, 8 and 9 recognize and bind to nucleic acids, such as viral dsRNA (TLR3), ssRNA (TLR7, TLR8) and unmethylated CpG DNA (TLR9). TLR4 recognizes and binds to fibronectin and LPS. TLR5 recognizes and binds to bacterial flagellin. Without wishing to be bound by any particular theory, it is believed that compounds of the disclosure do not result in general immunosuppression, but may exert their effects in a more selective and specific manner.

Another embodiment is a method of modulating (e.g., inhibiting) the activity of TLR3, TLR7, TLR8 and/or TLR9, comprising contacting a cell (e.g., a cell expressing TLR3, TLR7, TLR8 and/or TLR9; an immune cell) with a compound of the disclosure (e.g., a therapeutically effective amount of a compound of the disclosure). In some aspects, the compound of the disclosure selectively modulates (e.g., inhibits) the activity of TLR3, TLR7, TLR8 and/or TLR9, e.g., modulates (e.g., inhibits) the activity of TLR3, TLR7, TLR8 and/or TLR9 to a greater extent than it modulates the activity of TLRs 1, 2, 4, 5, 6 and/or 10. For example, modulation (e.g., inhibition) of the activity of TLR3, TLR7, TLR8 and/or TLR9 by a compound of the disclosure can be more than two-fold greater, e.g., more than five-fold, more than 10-fold, more than 25-fold or more than 100-fold greater, than modulation (e.g., inhibition) of the activity of TLRs 1, 2, 4, 5, 6 and/or 10 by the compound. In some aspects, the compound does not modulate (e.g., inhibit) the activity of TLRs 1, 2, 4, 5, 6 and/or 10 to a measurable extent.

In some aspects of the methods described herein, the cell is an immune cell, e.g., a T-cell, such as a regulatory T-cell, a natural killer (NK) cell, a macrophage, a neutrophil, a myeloid-derived suppressor cell or a dendritic cell. In some aspects, an immune cell is FoxP3+ and/or CD4+, such as a FoxP3+ and/or CD4+ T-cell. In some aspects, the immune cell is a B-cell, such as a regulatory B-cell. In some aspects, an immune cell (e.g., regulatory B-cell) is CD19+, CD71+, IgM+, CD24+, CD38+ and/or CD27+.

In some aspects of the methods described herein, the method is conducted in vitro. In other aspects of the methods described herein, the method is conducted in vivo. In some aspects, therefore, the cell (e.g., immune cell) is in a subject (e.g., a subject having a disease, disorder or condition described herein).

Another embodiment is a method of immunotolerizing a subject in need thereof (e.g., a subject having an autoimmune disorder, such as an autoimmune disorder described herein), comprising administering to the subject a therapeutically effective amount of a compound of the disclosure, e.g., in the form of a composition described herein.

Another embodiment is a method of immunotolerizing a subject in need thereof to an antigen (e.g., an antigenic therapy), comprising administering to the subject a therapeutically effective amount of a compound of the disclosure, e.g., in the form of a composition described herein. Some aspects comprise administering to the subject the antigen, or an immunogenic fragment thereof, and a therapeutically effective amount of a compound of the disclosure, e.g., in the form of a composition described herein. Some aspects comprise administering to the subject a composition described herein comprising the compound of the disclosure and the antigen, or an immunogenic fragment thereof, e.g., a composition comprising a plurality of lipid particles, wherein each lipid particle comprises the compound of the disclosure and the antigen, or an immunogenic fragment thereof. In some aspects, the antigen, or an immunogenic fragment thereof, and the compound of the disclosure are administered to the subject in separate formulations.

“Immunotolerizing,” as used herein, refers to diminishing and/or eliminating an immune response, e.g., to an antigen. An immune response can, for example, be evidenced by immunological hyperactivity, inflammatory cytokine release and/or activation of immune cells such as macrophages, neutrophils, eosinophils, T-cells and B-cells. “Immunotolerizing,” as used herein, contemplates, for example, decreasing immunological hyperactivity, inhibiting inflammatory cytokine release and/or inhibiting activation and/or neutralizing immune cells such as macrophages, neutrophils, eosinophils, T-cells and B-cells. In a clinical setting, immunotolerizing may be evidenced, for example, by reduced severity of autoimmune disease and/or improved activity of administered antigenic therapy.

Thus, the process of immunotolerizing can be viewed along a continuum that ranges from immunological hyperactivity to immunological hypoactivity to immunological non-responsiveness, e.g., to an antigen. “Immunotolerizing” contemplates incremental improvements along this continuum towards immunological non-responsiveness as well as inducing immunological hypoactivity or immunological non-responsiveness. In other words, immunotolerizing includes reducing the level of immune intolerance and inducing immune tolerance. In certain preferred embodiments described herein, the method induces immune tolerance.

In some aspects, a subject showing immune intolerance or an immune intolerant subject has a measurable immune response, e.g., to an antigen, such as measurable antibody production in response to an antigen. In some aspects, a subject showing immune tolerance or an immune tolerant subject, does not have a measurable immune response, e.g., to an antigen, such as measurable antibody production in response to an antigen. ELISA and/or activity assays, including those described herein, are known in the art, and can be used to measure antibody production indicative of immune intolerance.

In some autoimmune diseases, antibodies are not always present. Immune intolerance in such cases can be evident by clinical symptoms of autoimmune disease and/or the presence of self-reactive T-cells or B-cells and/or an increase in other inflammatory immune cells, such as neutrophils, eosinophils, etc. In some aspects, a subject showing immune intolerance or an immune intolerant subject (e.g., subject having an autoimmune disease, such as an autoimmune disease described herein) has a measurable cytokine response. For example, a subject having rheumatoid arthritis may have a measurable TNF-alpha response. In some aspects, a subject showing immune tolerance or an immune tolerant subject (e.g., subject having an autoimmune disease, such as an autoimmune disease described herein) does not have a measurable cytokine response.

Immunotolerizing can be achieved in a general or antigen-specific manner, resulting, for example, in general or antigen-specific immune tolerance (e.g., general or specific, acquired or adaptive, immune tolerance), respectively. Indicators of general immunotolerization include, for example: (a) absence and/or diminishment of immunological hyperactivity and/or anti-inflammatory cytokine release; (b) neutralization of immune cells such as macrophages, neutrophils, eosinophils, T-cells and B-cells; (c) an increase in number of regulatory T-cells and/or in the activity or level of tolerogenic T-cells (e.g., FoxP3+/CD4+T-cells; CD4+/CD25^{hi}/Foxp3+/CTLA4+/Tim3+/NRP1+/ICOS-T-cells; CD4+/CD25^{hi}/Foxp3+/CTLA4+/Tim3+ T-cells; and/or CD4+/CD25^{hi}/Foxp3+/CTLA4+/NRP1+/ICOS-T-cells); and/or (d) an increase in the number of regulatory B-cells (e.g., CD19+/CD71+/IgM+/CD24+/CD38+/CD27+ B-cells; and/or CD19+/CD71+/IgM+ B-cells). Indicators of antigen-specific immunotolerization include, for example: (a) an increase in the number of antigen-specific regulatory T-cells (e.g., CD$+/FoxP3+ T-cells; CD4+/CD25hi/Foxp3+/CTLA4+/Tim3+/NRP1+/ICOS-T-cells; CD4+/CD25hi/Foxp3+/CTLA4+/Tim3+ T-cells; and/or CD4+/CD25hi/Foxp3+/CTLA4+/NRP1+/ICOS-T-cells); (b) a decrease in antigen-specific antibody titer and/or number of B cells, including antigen-specific memory B cells; (c) a decrease in IL-6 and/or IL-17; (d) an increase in TGF-beta, IL-10, IL-35, CD40, CD80 and/or CD86; (e) hyporesponsiveness following re-challenge with an antigen; and/or (f) an increase in the number of antigen-specific regulatory B-cells (e.g., CD19+/CD71+/IgM+/CD24+/CD38+/CD27+ B-cells; and/or CD19+/CD71+/IgM+ B-cells). Techniques for evaluating these indicators are known in the art and described herein. For example, certain of the aforementioned indicators can be evaluated using culture conditions.

In autoimmune diseases, treatment with compounds of the disclosure leads to expansion of natural regulatory T-cells. Such treatment does not interfere with innate immune response, such as that mounted by innate immune cells responding to danger signals from pathogens, but results in general adaptive immunotolerization. Thus, immunotolerizing can be achieved herein without general innate immune suppression, such that, for example, a subject can still mount an innate immune response to an antigen (e.g., pathogen). In some aspects, immunotolerizing is general adaptive immunotolerization. In some aspects, immunotolerizing is antigen-specific, for example, resulting in reduced immune intolerance to a particular antigen(s) or immune tolerance to the particular antigen(s).

In some aspects, immunotolerizing is general, for example, resulting in generally reduced immune intolerance or general immune tolerance.

It will be understood that antigen-specific immunotolerizing can be achieved in accordance with the methods described herein not only by administering to a subject the specific antigen and a therapeutically effective amount of a compound of the disclosure or composition described herein, but also or alternatively by administering to a subject an immunogenic fragment of the specific antigen and a therapeutically effective amount of a compound of the disclosure or composition described herein.

As used herein, an “immunogenic fragment” of an antigen refers to a fragment of the antigen that induces an immune response to the antigen. An immunogenic fragment of an antigen may induce an immune response in a subject that is similar in extent to the immune response induced by the antigen itself, but need not induce the same extent of immune response as the antigen itself, so long as, when administered in accordance with the methods described herein, it has an immunotolerizing effect.

Another embodiment is a method of inhibiting or reducing an antigen-specific antibody titer in a subject, comprising administering to the subject the antigen, or an immunogenic fragment thereof, and a therapeutically effective amount of a compound of the disclosure, e.g., in the form of a composition described herein. Some aspects comprise administering to the subject a composition described herein comprising the compound of the disclosure and the antigen, or an immunogenic fragment thereof, e.g., a composition comprising a plurality of lipid particles, wherein each lipid particle comprises the compound of the disclosure and the antigen, or an immunogenic fragment thereof. In some aspects, the antigen, or an immunogenic fragment thereof, and the compound of the disclosure are administered to the subject in separate formulations.

In some aspects of a method described herein, the antigen is an allergen, such as a food allergen or latex allergen. Examples of food allergens include peanut allergen, such as Ara h I or Ara h II; walnut allergen, such as Jug r I; brazil nut allergen, such as albumin; shrimp allergen, such as Pen a I; egg allergen, such as ovomucoid; milk allergen, such as bovine β-lactoglobin; wheat gluten antigen, such as gliadin); and fish allergen, such as parvalbumins. An example of a latex allergen is Hey b 7. Other allergens include antigen E, or Amb a I (ragweed pollen); protein antigens from grass, such as Lol p 1 (grass); dust mite allergens, such as, Der pI and Der PII (dust mites); Fel d I (domestic cat); and protein antigens from tree pollen, such as Bet v1 (white birch), and Cry j 1 and Cry j 2 (Japanese cedar). The allergen source listed in parentheses next to each allergen indicates the source with which the indicated allergen is typically associated.

Another embodiment is a method of inducing a population of regulatory T-cells in a subject, comprising administering to the subject a therapeutically effective amount of a compound of the disclosure, e.g., in the form of a composition described herein. Some aspects further comprise administering to the subject an antigen, or an immunogenic fragment thereof, in response to which the population of regulatory T-cells is being induced. Some aspects comprise administering to the subject a composition described herein comprising the compound of the disclosure and the antigen, or an immunogenic fragment thereof, e.g., a composition comprising a plurality of lipid particles, wherein each lipid particle comprises the compound of the disclosure and the antigen, or an immunogenic fragment thereof. In some aspects, the antigen, or an immunogenic fragment thereof, and the compound of the disclosure are administered to the subject in separate formulations.

Without wishing to be bound by any particular theory, it is believed that the compounds of the disclosure induce a population of regulatory T-cells primarily by expanding the population of natural regulatory T-cells (nT_{regs}, e.g., regulatory T-cells which are FoxP3+/NRP1+). The compounds of the disclosure also induce or upregulate inducible regulatory T-cells (iT_{regs}, e.g., FoxP3+ T-cells, FoxP3+/TIM3+ T-cells). Accordingly, in some aspects, a method of inducing a population of regulatory T-cells is a method of expanding a population of natural regulatory T-cells (e.g., regulatory T-cells which are FoxP3+/NRP1+), for example, without substantially inducing inducible regulatory T-cells. Neuropilin-1 (Nrp1) expression can be used to distinguish between natural and inducible regulatory T-cells, for example, as described herein. Thus, in some aspects, a method of inducing a population of regulatory T-cells is a method of inducing a population of regulatory T-cells expressing Nrp1 (e.g., FoxP3+/NRP1+ T-cells) as, for example, by expanding a population of natural regulatory T-cells. Without wishing to be bound by any particular theory, it is expected that the ability to expand a population of natural regulatory T-cells (e.g., regulatory T-cells which are FoxP3+/NRP1+), for example, without substantially inducing inducible regulatory T-cells, will be beneficial in treating autoimmune diseases without effecting general immunosuppression.

In some aspects, regulatory T-cells are FoxP3+, e.g., FoxP3+/TIM3+, FoxP3+/NRP1+. Whether a regulatory T-cell is positive (+) or negative (−) for any of the aforementioned markers can be determined, for example, by flow cytometry analysis.

Another embodiment is a method of increasing the activity or level of tolerogenic T-cells in a subject, comprising administering to the subject a therapeutically effective amount of a compound of the disclosure, e.g., in the form of a composition described herein.

Another embodiment is a method of inducing a population of regulatory B-cells in a subject, comprising administering to the subject a therapeutically effective amount of a compound of the disclosure, e.g., in the form of a composition described herein. Some aspects further comprise administering to the subject an antigen, or an immunogenic fragment thereof, in response to which the population of regulatory B-cells is being induced. Some aspects comprise administering to the subject a composition described herein comprising the compound of the disclosure and the antigen, or an immunogenic fragment thereof, e.g., a composition comprising a plurality of lipid particles, wherein each lipid particle comprises the compound of the disclosure and the antigen, or an immunogenic fragment thereof. In some aspects, the antigen, or an immunogenic fragment thereof, and the compound of the disclosure are administered to the subject in separate formulations.

It has been found that the compounds of the disclosure increase expression of certain regulatory markers on B-cells, such as CD19, CD71 and IgM, and thereby induce a population of CD19+/CD71+/IgM+ B-cells. In some aspects, regulatory B-cells are CD19+, CD71+, IgM+, CD24+, CD38+ and/or CD27+, e.g., CD19+/CD71+/IgM+. Whether a regulatory B-cell is positive (+) or negative (−) for any of the aforementioned markers can be determined, for example, by flow cytometry analysis.

Another embodiment is a method of treating an autoimmune disorder in a subject, comprising administering to the subject a therapeutically effective amount of a compound of the disclosure, e.g., in the form of a composition described herein. It will be appreciated that in autoimmune disorders, it may be desirable to induce general adaptive immunotolerization (e.g., immune tolerance) as, for example, by inducing a population of regulatory T-cells, or specific immunotolerization (e.g., immune tolerance) as, for example, by immunotolerizing a subject to a self-antigen associated with the autoimmune disorder, or an immunogenic fragment thereof. Thus, in some aspects of a method of treating an autoimmune disorder, the method further comprises administering (e.g., co-administering) a self-antigen associated with the autoimmune disorder, or an immunogenic fragment thereof, to the subject. Some aspects comprise administering to the subject a composition described herein comprising the compound of the disclosure and the self-antigen, or an immunogenic fragment thereof, e.g., a composition comprising a plurality of lipid particles, wherein each lipid particle comprises the compound of the disclosure and the self-antigen, or an immunogenic fragment thereof. In some aspects, the self-antigen, or an immunogenic fragment thereof, and the compound of the disclosure are administered to the subject in separate formulations.

Specific examples of autoimmune disorders treatable according to the methods described herein include achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal and neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), hypogammalglobulinemia, IgA nephropathy, IgG4-related sclerosing disease, immune thrombocytopenia purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II and III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopeni purpura (TTP), thyroid eye disease (TED), Tolosa-Hunt syndrome (THS), transverse myelitis, Type 1 diabetes, ulcerative colitis (UC), undifferentiated connective tissue disease (UCTD), uveitis, vasculitis, vitiligo and Vogt-Koyanagi-Harada Disease.

In some aspects, the autoimmune disorder is a neurological autoimmune disorder. Examples of neurological autoimmune disorders include multiple sclerosis, neuromyelitis optica, myasthenia gravis, anti-myelin oligodendrocyte glycoprotein antibody disease (MOG), a MOG antibody-associated disorder (MOGAD, e.g., MOG-associated childhood demyelinating disease), autoimmune encephalitis, acute disseminated encephalomyelitis (ADEM), chronic meningitis, central nervous system vasculitis, Guillain-Barre syndrome, Hashimoto's thyroiditis, steroid responsive encephalopathy associated with autoimmune thyroiditis (SREAT), neurosarcoidosis, optic neuritis and transverse myelitis.

In some aspects, the autoimmune disorder is rheumatoid arthritis, systemic lupus erythematosus, inflammatory bowel disease (IBD), multiple sclerosis, type 1 diabetes mellitus, Guillain-Barre syndrome, chronic inflammatory demyelinating polyneuropathy, psoriasis, Graves' disease, Hashimoto's thyroiditis, myasthenia gravis or vasculitis. In some aspects, the autoimmune disorder is systemic lupus erythematosus. In some aspects, the autoimmune disorder is IBD.

In some aspects, the autoimmune disorder is multiple sclerosis, neuromyelitis optica, myelin oligodendrocyte glycoprotein antibody-associated disease (MOGAD), rheumatoid arthritis or myasthenia gravis. In some aspects, the autoimmune disorder is multiple sclerosis. In some aspects, the autoimmune disorder is neuromyelitis optica. In some aspects, the autoimmune disorder is MOGAD. In some aspects, the autoimmune disorder is rheumatoid arthritis. In some aspects, the autoimmune disorder is myasthenia gravis.

The clinical management of multiple sclerosis typically follows one of two paradigms: the escalation paradigm or the induction/maintenance paradigm. In the escalation paradigm, disease-modifying therapies (DMTs) of increasing efficacy and potency (and with greater risks of serious adverse events) are given following treatment failure with a lower-potency and lower-efficacy DMT. Typically, standard of care therapy in the escalation paradigm involves treatment with glatiramer acetate, interferon beta and/or teriflunimude, which is escalated upon treatment failure to fingolimod and/or dimethyl fumarate, which is further escalated upon treatment failure to natalizumab and/or anti-B-cell, which is yet further escalated upon treatment failure to alemtuzumab and/or mitoxantrone.

The induction/maintenance treatment paradigm for clinical management of multiple sclerosis includes an induction phase followed by a maintenance phase. Patients are treated with high potency DMTs to induce disease control during the induction phase, and are subsequently switched to a safer, lower potency DMT for maintenance therapy during the maintenance phase.

Disease-modifying therapies (DMT) used in the treatment of multiple sclerosis include interferon beta-1a (e.g., AVONEX®, REBIF®), interferon beta-1b (e.g., BETASERON®, EXTAVIA®), glatiramer acetate (e.g., COPAXONE®, GLATOPA®), ofatumumab (e.g., KESIMPTA®), peginterferon beta-1a (e.g., PLEGRIDY®), teriflunomide (e.g., AUBAGIO®), monomethyl fumarate (e.g., BAFIERTAMIM), dimethyl fumarate (e.g., TECFIDERA®), fingolimod (e.g., GILENYA®), cladribine (e.g., MAVENCLAD®), siponimod (e.g., MAYZENT®), ponesimob (e.g., PONVORY®), diroximel fumarate (e.g., VUMERITY®), ozanimob (e.g., ZEPOSIA®), alemtuzumab (e.g., LEMTRADA®), mitoxantrone (e.g., NOVANTRONE®), ocrelizumab (e.g., OCREVUS®) and natalizumab (e.g., TYSABRI®). Examples of high-potency DMTs include, but are not limited to, natalizumab, alemtuzumab, anti-B-cell and mitoxantrone. Examples of lower potency DMTs include, but are not limited to, glatiramer acetate, interferon beta, teriflunimide, DMF and fingolimob.

Natalizumab is a recombinant humanized IgG4_{κ} monoclonal antibody produced in murine myeloma cells. Natalizumab binds to the α4-subunit of α4β1 and α4β7 integrins expressed on the surface of all leukocytes except neutrophils, and inhibits the α4-mediated adhesion of leukocytes to their counter-receptor(s). Natalizumab injection is indicated as monotherapy for the treatment of relapsing forms of multiple sclerosis, including clinically isolated syndrome, relapsing-remitting disease, and active secondary progressive disease, in adults. Glatiramer acetate injection is indicated for the treatment of relapsing forms of multiple sclerosis, including clinically isolated syndrome, relapsing-remitting disease, and active secondary progressive disease, in adults. Dimethylfumarate for oral use is indicated for the treatment of relapsing forms of multiple sclerosis.

In some aspects, the multiple sclerosis is previously untreated. In alternative aspects, the multiple sclerosis is previously treated, e.g., with a standard of care therapy, such as natalizumab (TYSABRI®), glatiramer acetate and/or dimethyl fumarate, or a DMT.

In some aspects, multiple sclerosis is primary progressive multiple sclerosis (PPMS). In some aspects, multiple sclerosis is relapsing remitting multiple sclerosis (RRMS). In some aspects, multiple sclerosis is clinically isolated syndrome (CIS). In some aspects, multiple sclerosis is secondary progressive multiple sclerosis (SPMS).

In some aspects (e.g., wherein the autoimmune disease is multiple sclerosis), the method comprises administering to the subject a therapeutically effective amount of an induction therapy comprising a compound of the disclosure, e.g., in the form of a pharmaceutical composition. In some aspects (e.g., wherein the autoimmune disease is multiple sclerosis), the method comprises administering to the subject a therapeutically effective amount of a maintenance therapy comprising a compound of the disclosure, e.g., in the form of a pharmaceutical composition. In some aspects (e.g., wherein the autoimmune disease is multiple sclerosis), the method comprises administering to the subject a therapeutically effective amount of an induction therapy comprising a compound of the disclosure, e.g., in the form of a pharmaceutical composition, and (e.g., followed by) a therapeutically effective amount of a maintenance therapy comprising the compound of the disclosure, e.g., in the form of a pharmaceutical composition.

In some aspects (e.g., wherein the autoimmune disease is multiple sclerosis), a compound of the disclosure is administered in combination with a DMT, such as natalizumab and/or glatiramer acetate and/or dimethylfumarate, and, in some further aspects, the method further comprises administering a DMT, such as natalizumab and/or glatiramer acetate and/or dimethyl fumarate, to the subject.

In some aspects, an autoimmune disorder is previously untreated. In alternative aspects, an autoimmune disorder is previously treated, e.g., with a standard of care therapy, such as natalizumab (TYSABRI®) or glatiramer acetate for multiple sclerosis.

Examples of self-antigens associated with autoimmune disorders include thyroid stimulating hormone receptor of the thyroid gland (Grave's disease); thyroid antigens, such as thyroid peroxidase (Hashimoto's thyroiditis); β cell antigens, such as glutamic acid decarboxylase and insulin (type I diabetes); cytochrome P450 antigens (Addison's disease); myelin proteins, such as myelin basic protein (multiple sclerosis); uveal antigens (uveitis); gastric parietal cell antigens, such as H⁺/ATPase and intrinsic factor (pernicious anemia); transglutaminase (gluten enteropathy); myocardial cell proteins, such as myosin (myocarditis, rheumatic heart disease); platelet antigens, such as GP IIb/IIIa (idiopathic thrombocytopeni purpura); red blood cell membrane proteins (autoimmune hemolytic anemia); neutrophil membrane proteins (autoimmune neutropenia); basement membrane antigens, such as type IV collagen.alpha.3 chain, (Goodpasture's disease); intrahepatic bile duct/mitochondrial antigens, such as 2-oxoacid dehydrogenase complexes (primary biliary cirrhosis); hepatocyte antigens, such as cytochrome P450 and 206 (autoimmune hepatitis); acetylcholine receptors (myasthenia gravis); desmogleins (pemphigus and other bullous diseases). The disorder listed in parentheses next to each self-antigen indicates the autoimmune disorder with which the indicated self-antigen is typically associated.

Compounds of the disclosure and compositions described herein are expected to be useful adjunctive therapies in the context of antigenic therapy, such as gene therapy, e.g., as by inhibiting an undesirable immune response to the antigenic therapy and/or enabling dosing and/or repeat dosing of the antigenic therapy. Another embodiment is a method of treating a disease, disorder or condition in a subject in need thereof with an antigenic therapy, comprising administering to the subject a compound of the disclosure, e.g., in the form of a composition described herein. In some aspects, the compound of the disclosure is administered in an amount sufficient to immunotolerize the subject to the antigenic therapy. In some aspects, the method further comprises administering (e.g., co-administering) to the subject the antigenic therapy (e.g., a therapeutically effective amount of the antigenic therapy), for example, concurrently or sequentially with the compound of the disclosure. Some aspects comprise administering to the subject a composition described herein comprising the compound of the disclosure and the antigenic therapy, e.g., a composition comprising a plurality of lipid particles, wherein each lipid particle comprises the compound of the disclosure and the antigenic therapy. In some aspects, the antigenic therapy and the compound of the disclosure are administered to the subject in separate formulations.

In some aspects, the antigenic therapy is an antibody therapy (e.g., monoclonal antibody therapy), including chimeric, humanized and fully-human antibody therapies. Specific examples of antibody therapies include anti-tumor necrosis factor (anti-TNF) therapies, such as adalimumab (Humira®; for rheumatoid arthritis, juvenile idiopathic arthritis, psoriatic arthritis, ankylosing spondylitis, Crohn's disease, ulcerative colitis, plaque psoriasis, hidradenitis suppurativa, uveitis) and infliximab (Remicade®, for Crohn's disease, pediatric Crohn's disease, ulcerative colitis, pediatric ulcerative colitis, rheumatoid arthritis, ankylosing spondylitis, psoriatic arthritis, plaque psoriasis), golimumab (Simponi®, for rheumatoid arthritis, psoriatic arthritis, ankylosing spondylitis, polyarticular juvenile idiopathic arthritis), etanercept (Enbrel®, for rheumatoid arthritis, polyarticular juvenile idiopathic arthritis, psoriatic arthritis, ankylosing spondylitis, plaque psoriasis) and certolizumab pegol (Cimzia®, for Crohn's disease, rheumatoid arthritis, psoriatic arthritis, ankylosing spondylitis, non-radiographic axial spondyloarthritis, plaque psoriasis).

In some aspects, the antigenic therapy is a protein replacement therapy, for example, enzyme replacement therapy. Examples of protein replacement therapies include replacement therapies for coagulation disorders, such as Factor VIII and Factor IX for hemophilia A and B; enzyme replacement therapies for lysosomal storage diseases, such as alglucosidase alfa (Myozyme® and Lumizyme®) for Pompe disease; alpha-L-iduronidase for Hurler syndrome; and adenosine deaminase for adult-type adenosine deaminase deficiency.

In some aspects, the antigenic therapy is a gene therapy. Gene therapies typically work by one of the following three mechanisms: (1) by supplying a subject with a healthy copy of a disease-causing gene (as does voretigene neparvovec-rzyl (Luxturna®), for example); (2) by inactivating a disease-causing gene (as may ASOs and siRNA, for example); or (3) by introducing a gene into the body to help treat a disease. Gene therapies include DNA (e.g., antisense oligonucleotides (ASOs)) and/or RNA (e.g., siRNA), which can be delivered to a subject in vivo or ex vivo via a variety of products. In vivo gene delivery products include plasmid DNA, viral vectors (e.g., AAV, such as AAV9) and non-viral vectors, such as bacterial vectors or lipid nanoparticles. Other examples of non-viral vectors suitable for in vivo gene delivery include exosomes, polymeric particles, inorganic particles and lipid-polymer hybrid particles. Ex vivo gene delivery products include subject-derived cellular gene therapy products. Gene therapies also include gene editing technologies, such as CRISPR. Gene editing technologies, such as CRISPR, can conveniently be delivered to a subject via any of the products for in vivo gene delivery described herein. Specific examples of gene therapies include voretigene neparvovec-rzyl (Luxturna®, for retinal dystrophy); and onasemnogene abeparvovec-xioi (Zolgensma®, for pediatric spinal muscular atrophy).

In some aspects, the gene therapy comprises DNA and/or RNA and a viral vector. In some aspects, the viral vector is derived from an adeno-associated virus (AAV), such as a recombinant AAV. In some aspects, the AAV is AAV9. Other examples of viral vectors suitable for use in the context of the present disclosure include viral vectors derived from retrovirus, herpes virus, adenovirus, lentivirus, rabies virus, lentivirus, VSV, poxvirus (e.g., vaccinia virus, variola virus, canarypox), reovirus, semliki forest virus, yellow fever virus, sindbis virus, togavirus, baculovirus, bacteriophages, alphavirus, and flavavirus.

In some aspects, the antigenic therapy is a cellular therapy. An example of a cellular therapy is axicabtagene ciloleucel (Yescarta®, for relapsed or refractory large B-cell lymphoma). Another example of a cellular therapy is CAR-T cells.

Alloantigens, antigens present in some but not all individuals of a species and recognized as foreign by those that lack it, are often the basis for graft rejection reactions. Accordingly, another embodiment is a method of treating graft-versus-host disease in a subject in need thereof, comprising administering to the subject a therapeutically effective amount of compound of the disclosure or composition described herein.

Examples of alloantigens include, but are not limited to, major histocompatability complex (MHC) class I and class II antigens, minor histocompatability antigens, endothelial glycoproteins, such as blood group antigens, and carbohydrate determinants.

Another embodiment is a method for promoting wound healing in a subject in need thereof, comprising administering to the subject a therapeutically effective amount of compound of the disclosure, e.g., in the form of a composition described herein.

In some aspects of any of the methods described herein, the method further comprises administering an antigen, or an immunogenic fragment thereof, to the subject. In some further aspects, the antigen, or an immunogenic fragment thereof, and the compound of the disclosure are co-administered. For example, it is sometimes desired to induce antigen-specific immune tolerance (e.g., when a compound of the disclosure is being administered to immunotolerize a subject to an antigenic therapy). When antigen-specific immune tolerance is desired, the antigen, or an immunogenic fragment thereof, and the compound of the disclosure, e.g., in the form of a composition described herein, are preferably co-administered.

As used herein, “co-administer,” “co-administration” and the like refer to simultaneous or nearly simultaneous but sequential administration of two or more agents (e.g., a compound of the disclosure and an antigen) via the same route of administration at the same or nearly the same site on the body of a subject.

When co-administration is simultaneous (e.g., concurrent), a first agent (e.g., a compound of the disclosure) and a second agent (e.g., an additional therapeutic agent, an antigen, or an immunogenic fragment thereof) can be in separate formulations or the same formulation. Alternatively, the first and second agents can be administered sequentially as separate compositions. When co-administration is sequential, administration of subsequent composition(s) occurs within 24 hours of administration of a first composition and, preferably, within 12 hours, for example, within 10 hours, 5 hours, 4 hours, 3 hours, 2 hours, 60 minutes, 30 minutes, 15 minutes, 10 minutes or 5 minutes, of administration of the first composition. Typically, when co-administration is sequential, the administration of subsequent composition(s) follows immediately after completion of administration of the first composition, taking into account any manipulations that a clinician or subject administering the compositions may need to engage in to ready subsequent composition(s) for administration.

When co-administration is oral, the site of administration is the mouth, and the two or more agents being co-administered are administered at the same site, by mouth, whether or not they are given in a single formulation or separate formulations. When co-administration is by injection of two or more compositions, however, the site of administration is more typically nearly the same. In such situations, the anatomical sites of administration are typically less than 2 inches apart from one another, for example, less than about 0.5 inches, less than about 1 inch or less than about 1.5 inches from one another.

In some aspects, an antigen, or an immunogenic fragment thereof, and a compound of the disclosure are co-administered. In further aspects, administration of the antigen, or an immunogenic fragment thereof, precedes administration of the compound of the disclosure. In alternative further aspects, administration of the compound of the disclosure precedes administration of the antigen, or an immunogenic fragment thereof. In yet alternative further aspects, administration of the compound of the disclosure and the antigen, or an immunogenic fragment thereof, is concurrent.

Co-administration can occur by any route of administration described herein. In some aspects, a compound of the disclosure and an antigen, or an immunogenic fragment thereof, are co-administered orally. In some aspects, a compound of the disclosure and an antigen, or an immunogenic fragment thereof, are co-administered subcutaneously.

Without wishing to be bound by any particular theory, it is believed that it is sometimes desirable for a subject's immune system to encounter antigen and compound of the disclosure together, or for the antigen and compound of the disclosure to be “co-presented” to a subject's immune system. When a compound of the disclosure is co-administered with an antigen and the antigen is a protein, such as a protein replacement therapy, co-administration, as, for example, by injection of separate formulations of antigen and compound of the disclosure, is expected to provide for effective co-presentation of the compound of the disclosure and the antigen to a subject's immune system. In such applications, the compound of the disclosure may, but need not be, incorporated into a lipid particle. In preferred aspects of such applications, co-administration is subcutaneous, e.g., by injection. In applications involving delivery of a gene therapy (e.g., a gene therapy comprising DNA and/or RNA), it may be desirable, in order to promote effective co-presentation of the gene therapy and the compound of the disclosure to a subject's immune system, to formulate the gene therapy and the compound of the disclosure into lipid particles comprising the gene therapy and the compound of the disclosure. In preferred aspects, such particles are formulated for oral and/or parenteral (e.g., subcutaneous, intramuscular, intravenous, intradermal) administration, e.g., as by injection.

Also without wishing to be bound by any particular theory, it is believed that particular compounds of the disclosure identified herein embed in a liposome. Use of such compounds, e.g., in accordance with the methods disclosed herein, may be advantageous in aspects wherein effective co-presentation of the compound of the disclosure and the antigen to the subject's immune system is promoted by incorporation of compound of the disclosure and antigen into a lipid particle comprising the compound of the disclosure and the antigen.

A compound of the disclosure can also be administered in combination with one or more non-antigenic therapies to treat a disease, disorder or condition. When administered “in combination” with such non-antigenic therapies, the compound of the disclosure can be administered before, after or concurrently with the other therapy (ies) (e.g., additional therapeutic agent(s)). When administered simultaneously (e.g., concurrently), the compound of the disclosure and another therapy can be in separate formulations or the same formulation. Alternatively, the compound of the disclosure and another therapy can be administered sequentially, either at approximately the same time or at different times, as separate compositions. When the compound of the disclosure and the other therapy (e.g., therapeutic agent) are administered as separate formulations or compositions, the compound of the disclosure and the other therapy can be administered by the same route of administration or by different routes of administration. A skilled clinician can determine appropriate timing for administration of each therapy being used in combination (e.g., timing sufficient to allow an overlap of the pharmaceutical effects of the therapies). Typically, a combination therapy will provide beneficial effects of the drug combination in treating the diseases, conditions or disorders described herein.

In some aspects, a method described herein further comprises administering to the subject (e.g., a therapeutically effective amount of) an additional, non-antigenic therapy (ies), e.g., in combination with a compound of the disclosure or composition described herein. In some aspects, the compound of the disclosure or composition described herein is administered before the additional therapy (ies). In some aspects, the compound of the disclosure or composition described herein is administered after the additional therapy (ies). In some aspects, the compound of the disclosure or composition described herein is administered concurrently with the additional therapy (ies).

A therapeutically effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

A compound of the disclosure, composition described herein, antigen or other therapeutic agent can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound, antigen and/or therapeutic agent, respectively, and the particular disease to be treated. Administration can be local or systemic as indicated.

The preferred mode of administration can vary depending on the particular compound or agent.

In some aspects, administration (e.g., of a compound of the disclosure or composition described herein and/or an antigen) is oral. In some aspects, administration (e.g., of a compound of the disclosure or composition described herein and/or an antigen) is intravenous. In some aspects, administration (e.g., of a compound of the disclosure or composition described herein and/or an antigen) is subcutaneous.

A compound of the disclosure or composition described herein can be administered, in accordance with the methods disclosed herein, prophylactically, as when a subject with no known immune intolerance to an antigenic therapy is co-administered a compound of the disclosure or composition described herein with the antigenic therapy. A compound of the disclosure or composition described herein can also or alternatively be administered, in accordance with the methods disclosed herein, therapeutically, as when a subject has demonstrated immune intolerance to an antigen (e.g., an allergic reaction, graft rejection). Accordingly, in some aspects, a subject has no known immune intolerance to an antigen, for example, because the subject is naïve to the antigen. In some aspects, a subject has no known immune intolerance to an antigen after having been administered and/or exposed to the antigen. In some aspects, a subject is immune intolerant to an antigen, for example, developed immune intolerance after having been administered and/or exposed to the antigen or is inherently immune intolerant to the antigen.

A compound of the disclosure or composition described herein can be administered (e.g., co-administered), in accordance with the methods disclosed herein, upon a first exposure to an antigen, as when a compound of the disclosure or composition described herein is administered with a first dose of an antigenic therapy. Also or alternatively, a compound of the disclosure or composition described herein can be administered (e.g., co-administered), in accordance with the methods disclosed herein, upon a second or further additional exposure to an antigen, as when a compound of the disclosure or composition described herein is administered with a second or further additional dose (e.g., a repeat dose) of an antigenic therapy.

The methods described herein are intended to reduce immune intolerance to an antigen for an extended period of time, for example, a period of time necessary to treat a disease, disorder or condition with an antigenic therapy described herein, for life of a subject. Accordingly, in some aspects of the methods described herein, the method further comprises administering the antigen, or an immunogenic fragment thereof (e.g., antigenic therapy, such as a therapeutically effective amount of the antigenic therapy), to the subject in the absence of the compound of the disclosure or composition described herein.

However, a subject's immune intolerance may increase over time following a method described herein, e.g., following subsequent exposure(s) to the antigen. In such cases, the methods described herein can be repeated, for example, as a “booster” vaccine is repeated, to re-immunotolerize the subject to the antigen.

A compound of the disclosure or other therapeutic agent described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. In some embodiments, administration (e.g., of a compound of the disclosure) is oral. In some embodiments, administration (e.g., of a compound of the disclosure) is intravenous. The preferred mode of administration can vary depending on the particular compound or agent. Typically, a compound of the disclosure or other therapeutic agent will be administered from about 1 to about 6 (e.g., 1, 2, 3, 4, 5 or 6) times per day, also or alternatively, as an infusion (e.g., a continuous infusion). In some aspects, the administration (e.g., of a compound of the disclosure is QD or BID (e.g., QD)). In some aspects, the administration (e.g., of a compound of the disclosure) is daily.

It is shown herein that orally administered liposomes, such as those described herein, can reach the lymph node, and colocalize with immune cells, including B-cell and T-cells, in the lymph node. Accordingly, also provided herein is a method of delivering a therapeutic agent (e.g., a compound of the disclosure) to a lymph node of a subject (e.g., a subject in need thereof), comprising orally administering to the subject a therapeutically effective amount of a composition comprising a plurality of lipid particles (e.g., solid lipid particles), wherein each lipid particle comprises at least one phospholipid (e.g., a phospholipid containing a C₄-C₃₀ acyl chain, such as a saturated C₄-C₃₀ acyl chain, as in dimyristoylphosphatidylcholine (DMPC)) and a therapeutic agent that can embed in a lipid bilayer of the lipid particle (e.g., a compound of the disclosure).

A compound of the disclosure or other therapeutic agent can be administered in a dosage ranging from about 0.001 mg/kg to about 100 mg/kg of body weight or, alternatively, in a dosage ranging from about 1 mg/dose to about 5,000 mg/dose, every 4 to 120 hours, or according to the requirements of the particular agent. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. In some aspects, a suitable dosage (e.g., daily dosage) is from about 0.1 mg/kg to about 10 mg/kg, e.g., from about 0.1 mg/kg to about 5 mg/kg, from about 0.1 mg/kg to about 2.5 mg/kg or about 0.2 mg/kg to about 2.4 mg/kg, body weight per treatment. Suitable dosages can be from about 0.001 mg/dose to about 100 mg/dose, from about 0.01 mg/dose to about 100 mg/dose, from about 0.1 mg/dose to about 50 mg/dose, from about 0.1 mg/dose to about 10 mg/dose, from about 0.5 mg/dose to about 50 mg/dose, from about 1 mg/dose to about 10,000 mg/dose, from about 1 mg/dose to about 7,500 mg/dose, from about 1 mg/dose to about 5,000 mg/dose, from about 10 mg/dose to about 2,500 mg/dose or from about 100 mg/dose to about 1,000 mg/dose. In some aspects, a suitable dosage (e.g., daily dosage) is from about 10 mg/dose to about 1,000 mg/dose, e.g., from about 15 mg/dose to about 1,000 mg/dose, from about 10 mg/dose to about 500 mg/dose, from about 10 mg/dose to about 250 mg/dose or from about 15 mg/dose to about 150 mg/dose.

Doses lower or higher than those recited above may be required. Specific dosage and treatment regimens for any particular patient will depend, for example, upon a variety of factors, such as the activity of the specific agent employed, the age, body weight, general health status, sex, diet, time of administration, rate of excretion, drug combination, the severity and course of the disease, condition or symptoms, the subject's disposition to the disease, condition or symptoms, and the judgment of the treating physician. Determining the dosage for a particular agent, subject and disease, disorder or condition is within the abilities of one of skill in the art.

### Exemplification

### Example 1. Synthesis of Compound 1 and Compound 2

The syntheses of Compound 1 and Compound 2 used the following Boc-Dap-OH as a common starting material:

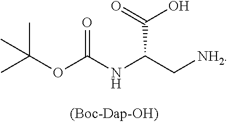

Compound 1: (S)-2-amino-3-(((hexadecyloxy) carbonyl)amino) propanoic acid

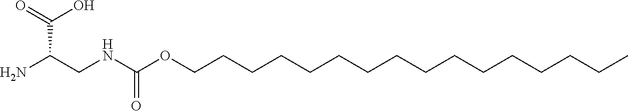 [SMILES: CCCCCCCCCCCCCCCCOC(=O)NC[C@H](N)C(=O)O]

Boc-Dap-OH (0.500 g) was dissolved in a 10% sodium carbonate solution (7 mL) at 0° C., and a solution of cetylchloroformate (0.866 g) in dioxane (7 mL) was added dropwise at 0° C. The reaction mixture was stirred at 0° C. for 1 hour and then stirred at room temperature (RT) for 1.5 hours. The reaction was then quenched with 60 mL water. The heterogenous mixture was extracted with ether. The solid was suspended in between the aqueous layer and the ether layer. The aqueous layer was separated, and the solid was filtered and washed with excess ether. The solid was suspended in water, acidified to pH 1, and extracted immediately with EtOAc (2×35 mL). The separated organic layer was washed with water (3×20 mL) and brine (15 mL) and dried over Na₂SO₄. The solvent was removed under reduced pressure to yield(S)-2-((tert-butoxycarbonyl)amino)-3-(((hexadecyloxy) carbonyl)amino) propanoic acid.

To generate(S)-2-amino-3-(((hexadecyloxy) carbonyl)amino) propanoic acid HCl salt, a solution of(S)-2-((tert-butoxycarbonyl)amino)-3-(((hexadecyloxy) carbonyl)amino) propanoic acid (0.423 g) in 4N HCl/Dioxane (7 mL) was stirred at 0° C. for 2.5 hours. The solvent was subsequently removed under reduced pressure to yield(S)-2-((tert-butoxycarbonyl)amino)-3-(((hexadecyloxy) carbonyl)amino) propanoic acid. The trace solvent was further removed at 70° C. under vacuum for 5 hours.

Compound 2: (S)-2-amino-3-stearamidopropanoic acid

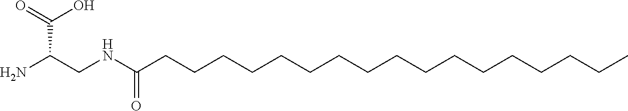 [SMILES: CCCCCCCCCCCCCCCCCC(=O)NC[C@H](N)C(=O)O]

Boc-Dap-OH (0.500 g) was dissolved in 10% sodium carbonate solution (7 mL) at 0° C., and a solution of stearoyl chloride (0.86 g) in dioxane (7 mL) was added dropwise at 0° C. The reaction mixture, thickened with a white precipitate, was stirred at 0° C. for 10 minutes and then stirred at RT for 2 hours. The reaction was quenched with 60 mL water. Ether was added, and the solution became emulsified. More ether was added. The emulsion was washed with ether (3×25 mL). When the emulsion was acidified to pH 1, the ether layer separated and the aqueous layer was turbid. The ether layer was separated and dried over Na₂SO₄. The solvent was removed under reduced pressure to yield(S)-2-((tert-butoxycarbonyl)amino)-3-(stearamido) propanoic acid.

To generate(S)-2-amino-3-stearamidopropanoic acid HCl salt, a solution of(S)-2-((tert-butoxycarbonyl)amino)-3-(stearamido) propanoic acid (0.502 g) in 4N HCl/Dioxane (9 mL) was stirred at 0° C. for 2.5 hours. Ether was added. The precipitated compound was filtered and washed with excess ether. The trace solvent was removed under high vacuum overnight.

### Example 2. Liposome Synthesis

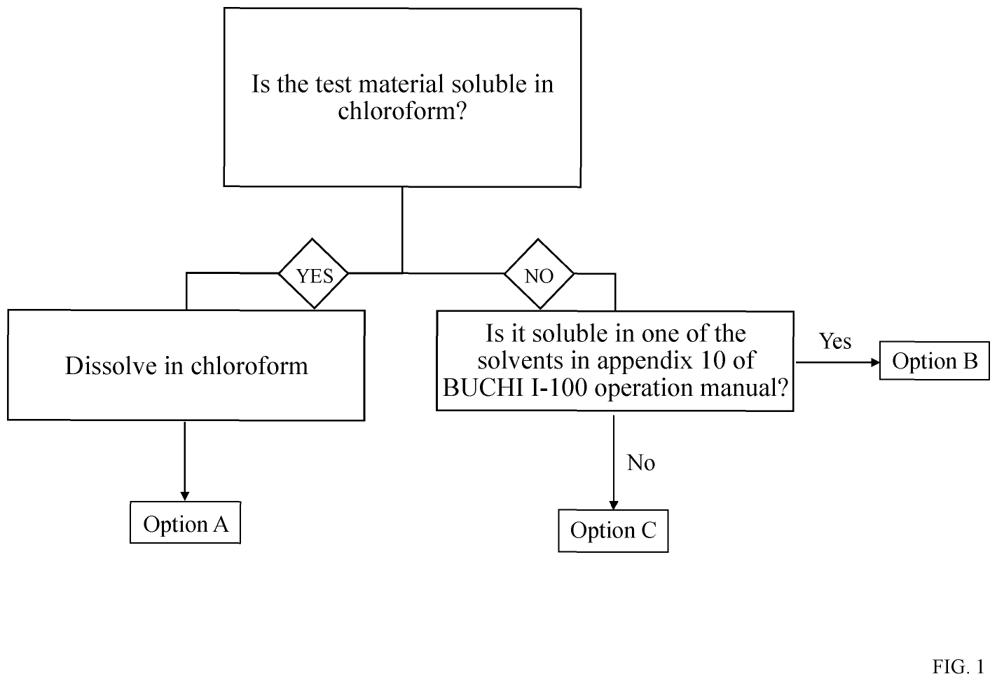

On the day before liposome preparation, the route of synthesis was determined based on the decision tree depicted in FIG. 1. In options A and B, the active ingredient was dissolved in an appropriate solvent (e.g., chloroform (Sigma-Aldrich, #650498-1L, St. Louis, MO)) and stored in an amber glass vials at −80° C. In option C, a stock solution was not made until the day of liposome preparation.

On the day of liposome preparation, the amount of the base lipid and active ingredient were calculated. In option A, if the test material was dissolved in chloroform, the volume of test material needed to obtain the desired molar concentration of test material was calculated. Also calculated was the amount of base lipid needed to make up the remaining molar ratio. For example, for a 30:70 molar ratio of test material:base lipid, the amount of base lipid is the amount of test material multiplied by 70 and divided by 30. The proper volumes of the test material and base lipid were aliquoted into a 5- or 15-ml round bottom flask, and 1 to 2 ml chloroform was added.

In option B, if the test material and the base lipid were dissolved in non-miscible solvents or the two solvents require different temperature and vacuum settings (e.g., DMSO and chloroform), the volume of test material and amount of base carrier lipid needed to obtain the desired molar concentration were calculated as in option A. Evaporation started with the solvent requiring higher temperature and higher vacuum before the material was added to the solvent requiring a lower temperature and vacuum. The temperature and vacuum were adjusted accordingly.

In option C, if the test material was water-soluble, the liposome was made up of 100% base lipid. The amount of base lipid was determined based on the amount of test material to be loaded on the fully synthesized liposome. For example, for a 30:70 molar ratio of test material:base lipid, the amount of the test material was multiplied by 100 and divided by 30 to obtain the amount of base lipid. Proper volumes of base lipid were aliquoted into a 5- or 15-ml round bottom flask, and 1 to 2 ml of chloroform was added.

A round-bottomed flask was attached to a rotary evaporator (R100 rotary evaporator with V100 and 1100 vacuum pump and interface, BÜCHI Labortechnik AG, Flawil, Switzerland), and a portion of the flask was submerged in a water bath by lowering the adjustable arm. The vacuum pump was turn on. To rotate the round-bottomed flask, the rotating shaft was turned on, and the speed was set to “3.” The vacuum was reduced as needed to avoid bubbling/boiling of the solvent in the flask. Once a uniform dry film was formed, the rotating shaft was turned off. In Option B, the second solvent containing the second material was added, and the above steps were repeated.

For rehydration, an appropriate aqueous buffer (e.g., PBS (Thermo Fisher Scientific (Gibco), #10010049, Waltham, MA)) was added (1 ml in Option A or B; 0.5 ml in Option C, and the pH was adjusted as needed). The round-bottomed flask was vortexed until the solution became milky/turbid. The flask was attached back to the rotary evaporator, and the arm was lowered so that the bottom of the flask was submerged in the water bath. The rotating shaft was turned on, to setting “3,” but the pump was not turned on. After 15 minutes, the lipid film was fully hydrated, and the liposomes were formed and ready for sizing.

For extrusion and sizing, the manual extruder (Avanti Corporation, Alexandria, VA, AVANTI manual extruder: 610023-1EA, PC Membranes 0.8 μm: 610009-1EA) was assembled. The total volume (not exceeding 1 ml) was aspirated into one of two syringes supplied with the manual extruder. The liposomal preparation was extruded 20 times. The final extruded liposomal formulation was transferred, and the volume was made up as needed to obtain the final desired molar concentration of the test material.

### Example 3. The Effect of Acyl Chain Length and Saturation on Lymphatic Uptake and Immune Cell Co-Localization of Orally Administered Compound 2 Containing Liposomes

The oral route is a patient-friendly route of administration. It also offers a unique opportunity to target the immune system via the mesenteric lymph nodes, which could prove useful in administering immune-modulating therapies. Compound 2, a novel amphiphilic molecule designed to span the lipid bi-layer of a liposome membrane, is thought to be a TIM agonist. A preliminary screen with Compound 2 indicated that it is pharmacologically active and can indeed induce a tolerogenic immune response in vitro and in vivo. Compound 2 can increase FoxP3⁺/CD4⁺ T-cells in vivo (Example 5) and FoxP3⁺/TIM3⁺ CD4 T-cells in vitro (Example 4).

The goal was to optimize a liposomal formulation that could target both the follicles of the cortex for B-cell engagement and the paracortex for T-cell engagement. Natural phospholipids with phosphatidylcholine (PC) headgroup were chosen as the base lipid. This was due, in part, to the neutral charge of PC. The effects of different acyl chain lengths and saturation levels on lymphatic uptake and immune cell co-localization after oral administration of liposomes were evaluated in vivo.

The materials and reagents used in this experiment are listed in Table 1.

TABLE 1

| | |
|---|---|
| Mice BC57BL6 | The Jackson Laboratory, |
| | Bar Harbor, ME |
| 1,1′-Dioctadecyl-3,3,3′,3′-Tetramethylindodicarbocyanine | Thermo Fisher Scientific, |
| Perchlorate (DilC₁₈(5)) far-red fluorescent dye | Waltham, MA |
| 1,2-dimyristoyl-sn-glycero-3-phosphocholine 14:0 (DMPC) | AVANTI Polar Lipids |
| 1,2-dioleoyl-sn-glycero-3-phosphocholine 18:1 9-Cis PC (DOPC) | Inc., Birmingham, AL |
| 1,2-distearoyl-sn-glycero-3-phosphocholine 18:0 (DSPC) | |
| 1-palmitoyl-2-oleoyl-glycero-3-phosphocholine 16:0-18:1 (POPC) | |
| fluorochrome-conjugated (FITC)-CD45R | Thermo Fisher Scientific |
| | (Invitrogen), Waltham, |
| fluorochrome-conjugated (FITC)- TCR-β | MA |
| DAPI mounting medium | Vector Laboratories, |
| | Burlingame, CA |

For in vitro optimization of lipid: DilC18 (5)-DS fluorescent-dye ratio, DMPC liposomes (AVANTI Polar Lipids Inc., Birmingham, AL) were prepared as described in Example 2. The lipid film was hydrated with PBS containing different concentrations of the DilC₁₈ (5)-DS fluorescent dye (100 nM to 2 μM). Fluorescence intensity was measured in a 96-well plate at Ex/Em 650 nm/670 nm to identify the lowest concentration of DilC18 (5) needed to obtain a reliable fluorescence measurement.

The test material was prepared as follows. Compound 2 was dissolved in DMSO. Four different formulations, one for each lipid in Table 1, were prepared at a molar ratio of 30:70 Compound 2:Lipid as described in Example 2. PBS containing 100 nM of DilC₁₈ (5) dye was used to hydrate the lipid film. The fluorescence intensity was measured in a 96-well plate at Ex/Em 650 nm/670 nm for each preparation.

Animals (n=3 per group) were dosed with 200 μl of 160 μM of each fluorescent liposome by oral gavage. All animals received the same dose of lipids regardless of the fluorescence intensity. The control group received a 200 μl oral gavage of PBS containing 100 nM of DilC₁₈ (5). To quantify the lymphatic uptake and immune cell co-localization of each liposomal formulation, animals were sacrificed 60 minutes after oral gavage. The mesenteric lymph node was extracted and preserved in optimal cutting temperature (OCT) compound for sectioning, labeling, and imaging.

To stain T and B cells, mesenteric lymph nodes were excised 60 minutes post oral gavage and snap-frozen in the OCT compound. Ten-micrometer sections were cut at −20° C., and the frozen sections were fixed in 2% paraformaldehyde (PFA) for 15 minutes at room temperature. The sections were washed in 1x PBS followed by incubation with fluorochrome-conjugated (FITC) primary antibody at 1:100 dilution of either CD45R or TCR-β in 1x PBS overnight at 4° C. On the next day, the slides were washed three times in 1×PBS and mounted with DAPI containing mounting medium.

For fluorescence imaging, the stained sections of mesenteric lymph nodes were visualized under an epifluorescence microscope (Nikon E-800; Nikon Inc., Melville, NY). Imaging was performed using channel Cy5 (Ex/Em 650 nm/670 nm) for the DilC-18 (5)-DS fluorescent dye (red), channel FITC (Ex/Em 494 nm/518 nm) for CD45R or TCR-β (green), and channel DAPI (Ex/Em 358 nm/461 nm) for the nucleus (blue).

Visual inspection of immunofluorescence gave a very good qualitative sense of liposome disposition within lymph node sections and level of colocalization with B- and T-cells. However, visual inspection did not account for differences in fluorescence intensity of each liposome, as seen in Table 2. Furthermore, the visual inspection did not allow for an objective differentiation between the liposomal formulations. For an objective evaluation, colocalization analysis and percent colocalization were performed.

Colocalization of liposomes with B- and T-cells was determined in ImageJ. Images from each channel (FITC, Cy5, and DAPI) were merged. The percent area of colocalization was calculated by thresholding the merged images to the yellow color spectrum, representing areas where green (immune cells) and red (liposomes) were colocalized. The quantification from the image analysis was normalized by subtracting the background obtained from the control group (treated with only free DilC₋₁₈ (5)-DS dye). Correction for different fluorescence intensities of each liposome was performed by normalizing the result of image thresholding to the fluorescent intensity (FI) of each liposome as described earlier to obtain corrected mean FI for each section.

Immunofluorescence staining was expected to yield high variability from one section to another because sections of tissue were not homogenous. To determine the effectiveness of liposome/immune-cell interaction, the mean FI of the colocalized area was normalized to the mean FI of immune cells using the following formula:

$$colocalization = \frac{corrected mean FI for each section}{mean FI for immune cells in each section}$$

Colocalization analysis was performed for all sections in all groups except the free dye control group since free dye/immune cell colocalization was not observed in those sections.

Fluorescence intensity measurement of DilC₁₈ (5) in PBS at concentrations ranging from 100 nM to 2 μM confirmed the dye's poor fluorescence in aqueous media compared to its fluorescence once incorporated into liposomes (Table 2). The relative fluorescence intensity (RFI) of the free dye remained low in the concentration range from 100 nM until 1 μM. At 2 μM, the fluorescence of DilC₁₈ (5) increased in PBS but remained lower than the liposomal DilC₁₈ (5). The RFI of DilC₁₈ (5) in liposomal formulation increased by >2000% in the range between 100 and 500 nM compared to the RFI of the dye in PBS. This was even greater in the liposomal formulation with 1 μM dye. The RFI decreased in the 2 μM liposomal formulation, which could be a result of self-quenching or poor incorporation of the dye into the liposome at such a high concentration (Table 2). The result of this screen suggests that 100 nM of DilC₁₈ (5) is enough to detect the fluorescence of liposomes and, further, the poor RFI of the dye in PBS at that concentration suggests that a purification step to remove free dye is not needed.

TABLE 2
Relative Fluorescence Intensity (RFI) as a
Function of Dye Concentration

| | RFI | |
|---|---|---|
| Dye conc. | Free (PBS) | Liposomal (DMPC) |
| 100 nM | 0.19 | 5.37 |
| 200 nM | 0.12 | 7.05 |
| 500 nM | 0.02 | 5.39 |
| 1 μM | 0.39 | 40.08 |
| 2 μM | 6.83 | 12.75 |

The fluorescence intensity measurements of each liposomal formulation revealed the differences between them. Liposomes made with the unsaturated DOPC and POPC had a higher RFI (73.2 and 61.8, respectively) than liposomes made with the saturated DMPC and DSPC (35.8 and 20.4, respectively) (Table 3).

TABLE 3
Relative Fluorescence Intensity (RFI) by liposome

| Formulation | DMPC | DOPC | DSPC | POPC |
|---|---|---|---|---|
| RFI | 35.2 | 72.8 | 20.6 | 60.2 |
| | 36.6 | 73.4 | 20.6 | 60.3 |
| | 35.7 | 73.3 | 20.0 | 64.9 |
| Mean | 35.8 | 73.2 | 20.4 | 61.8 |
| SD | 0.7 | 0.3 | 0.4 | 2.7 |

Sectioning and immunofluorescence staining were successful for all samples except for one sample from the free dye control group. Upon staining, it became apparent that this sample was for mesenteric fat, not a mesenteric lymph node. All other sections were stained successfully for image analysis.

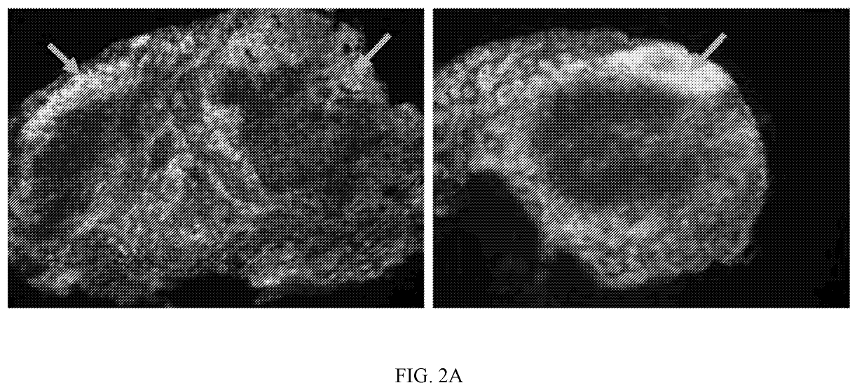

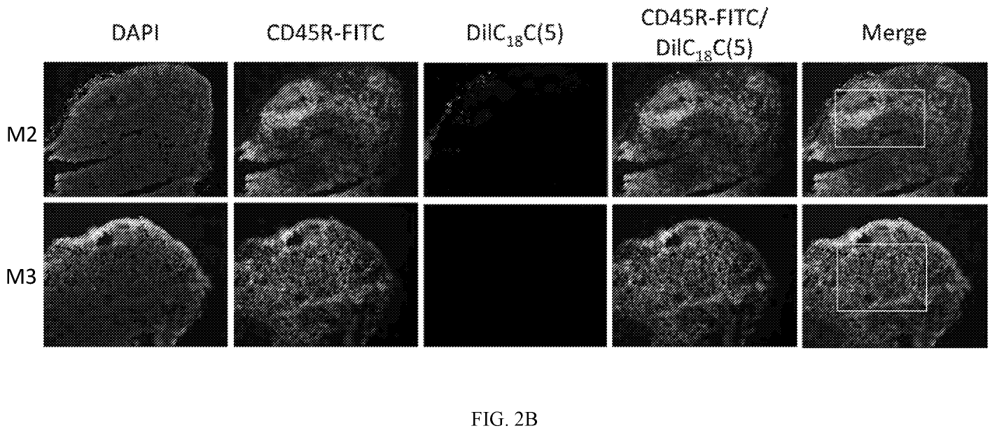

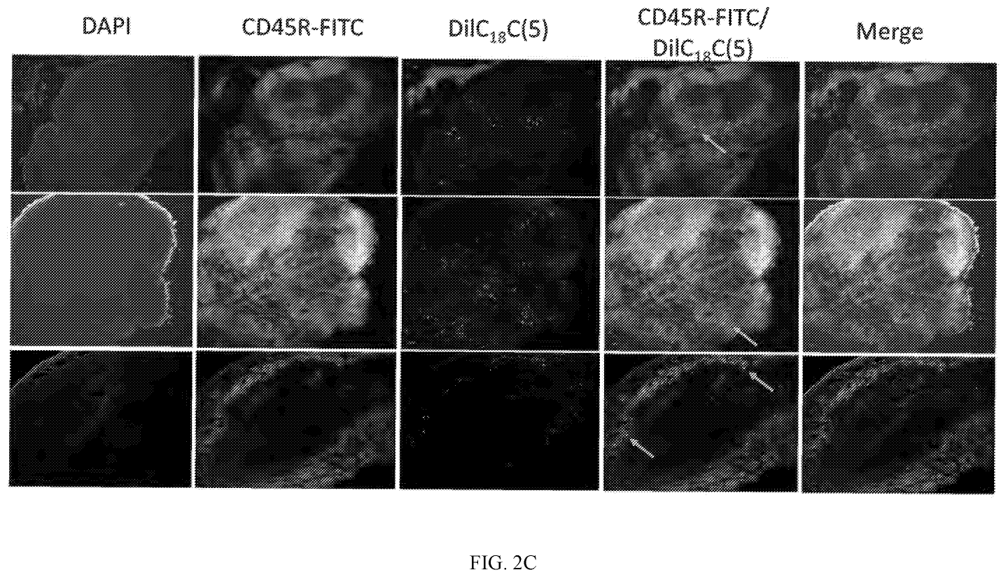

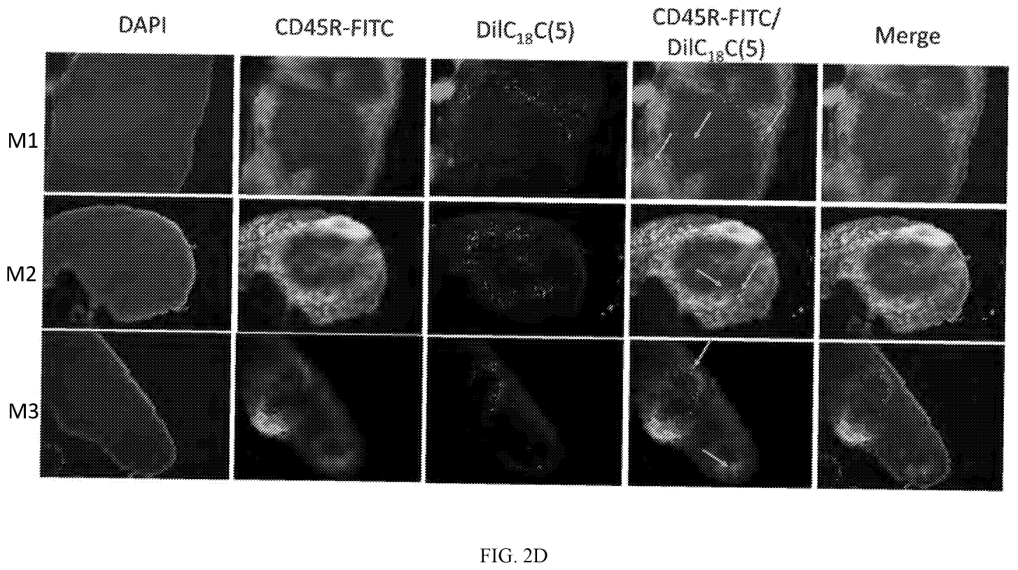

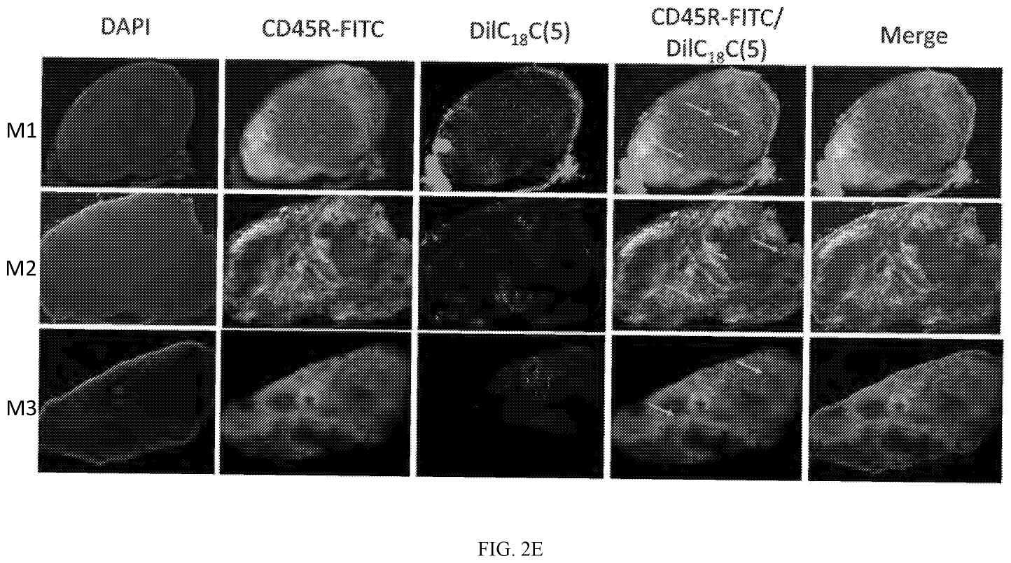

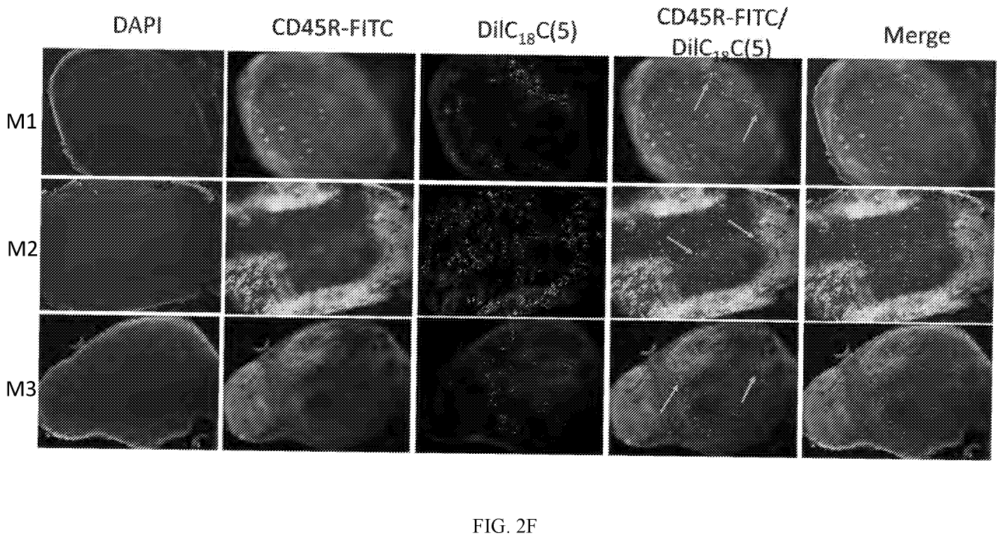

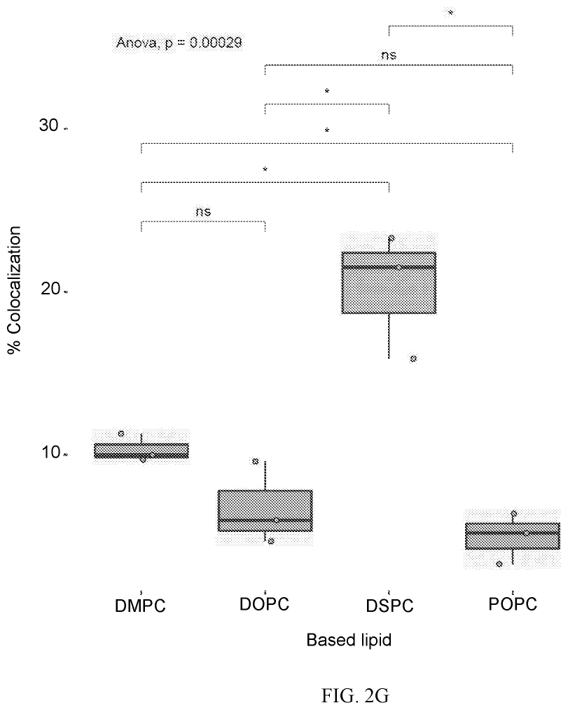

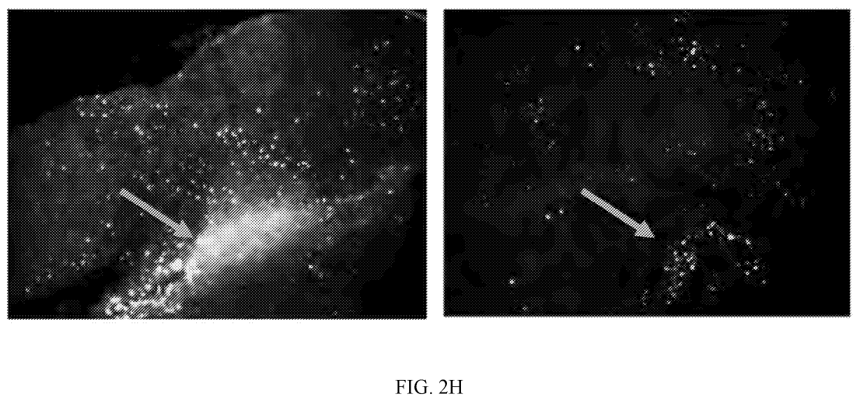

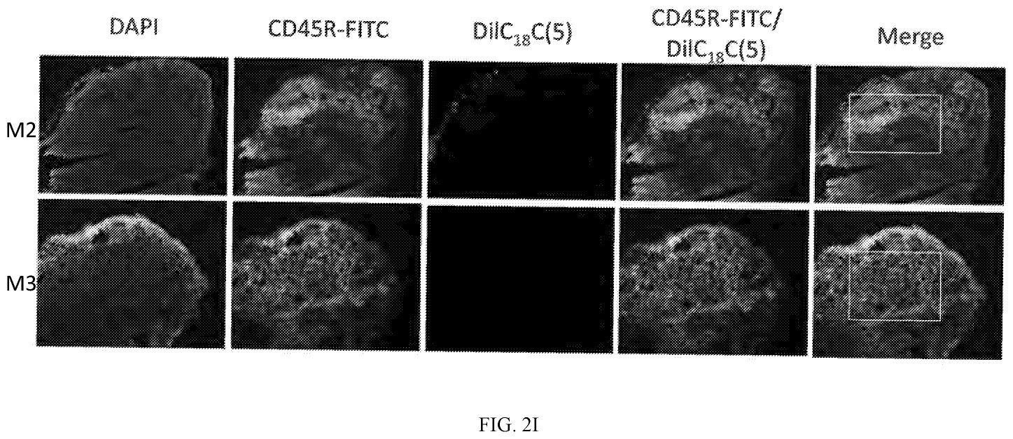

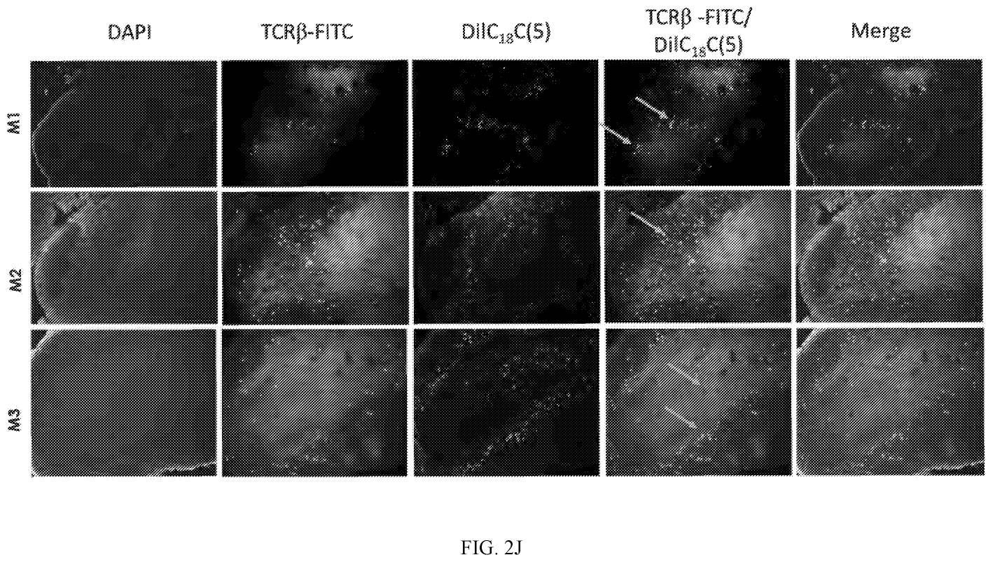

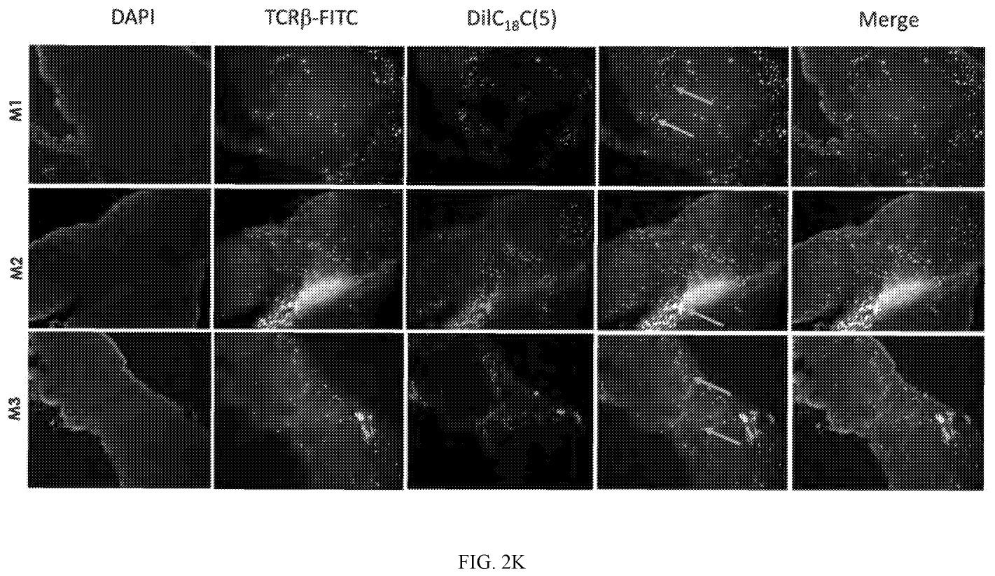

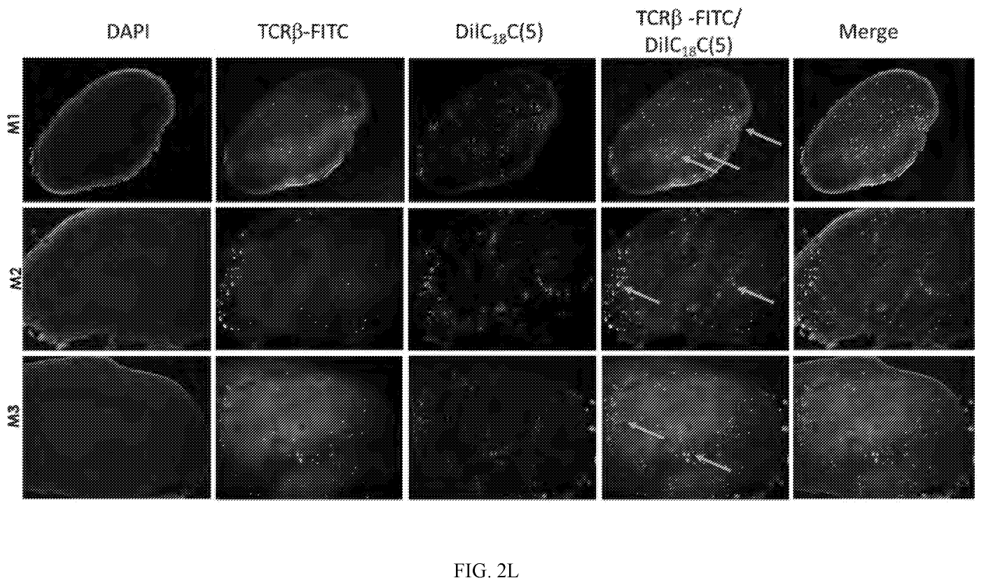

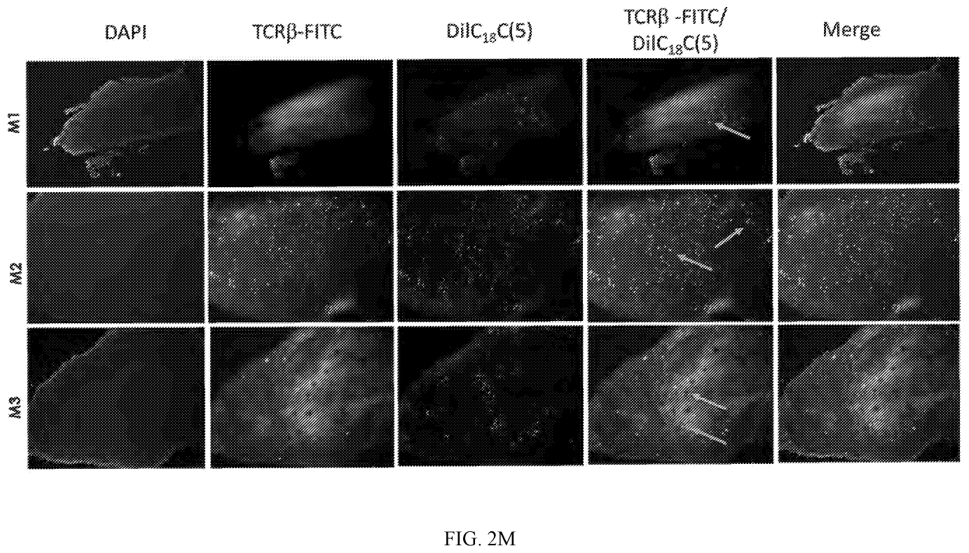

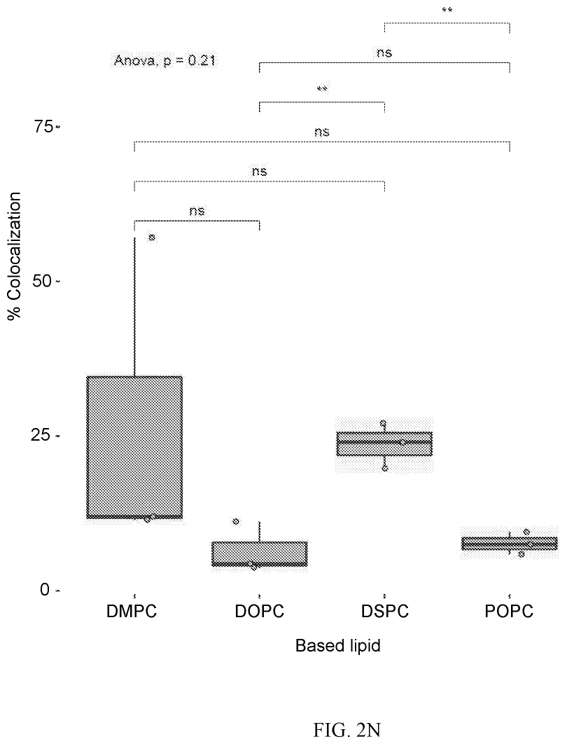

Immunofluorescence staining was performed to evaluate colocalization of B-cells and liposomes. FITC-conjugated anti-CD45R was observed in all samples, indicating the presence of B-cells. Both mature (bright) and immature (dim) B-cells were seen in all sections from all treatment groups. In some sections, B-cells were clustered, presumably in the cortex of the lymph node (arrows in FIG. 2A), around dark centers representing areas of the lymph node void of B-cells (e.g., paracortex or medulla).

The lymph node uptake and B-cell colocalization 60 minutes after an oral dose of 200 μl of PBS containing 100 nM of DilC18 (5) were evaluated. DAPI staining shows the general structure of the lymph node section. CD45R-FITC shows B-cell localization. Cy5 channel, which detects DilC18 (5) fluorescence, showed a signal in one sample, mainly concentrated on the outer periphery of the section (see FIG. 2B, M2, DilC18 (5) panel). However, no colocalization was observed (FIG. 2B, M2, CD45R-FITC/DilC18 (5) panel). No DilC18 (5) was detected in the lymph node from the other sample (FIG. 2B, M3).

The lymph node uptake and B-cell colocalization 60 minutes after a 200-μl oral dose of 160 μM of Compound 2/DMPC DilC18 (5)-DS labeled liposomes were evaluated. DAPI, staining shows the general structure of the lymph node section. CD45R-FITC shows B-cell localization. Cy5 channel, which detects DilC18 (5) fluorescence, showed DMPC liposome penetration into the lymph node. Merging FITC and Cy5 channels showed colocalization of B-cells and DMPC liposomes (see arrows in FIG. 2C, the CD45R-FITC/DilC₁₈ (5) panel). DMPC colocalization occurred mainly in the bright fluorescent region, suggesting colocalization with mature B-cells.

The lymph node uptake and B-cell colocalization 60 minutes after a 200-μl oral dose of 160 μM of Compound 2/DOPC DilC18 (5)-DS labeled liposomes were evaluated. DAPI staining was used to show the general structure of the lymph node section. CD45R-FITC was used to show B-cell localization. Cy5 channel, which detects DilC18 (5) fluorescence, was used to show DOPC liposome penetration into the lymph node. Merging FITC and Cy5 channels showed colocalization of B-cells and DOPC liposomes. Unlike DMPC, DOPC seemed to penetrate deeper into the lymph node. Colocalization was seen in both the bright and dim fluorescent regions, suggesting colocalization with both mature and immature B-cells (see arrows in FIG. 2D, the CD45R-FITC/DilC₁₈ (5) panel).

The lymph node uptake and B-cell colocalization 60 min after a 200 μl oral dose of 160 μM of Compound 2/DSPC DilC₁₈ (5)-DS labeled liposomes were evaluated. DAPI staining shows the general structure of the lymph node section. CD45R-FITC shows B-cell localization. Cy5 channel, which detects DilC₁₈ (5) fluorescence, showed DSPC liposome penetration into the lymph node. Merging FITC and Cy5 showed colocalization of B-cells and DSPC liposomes. Similar to DOPC, DSPC seemed to penetrate deep into the lymph node. Colocalization can be seen in both the bright and dim fluorescent region suggesting colocalization with both mature and immature B-cells (see arrows in FIG. 2E, CD45R-FITC/DilC₁₈ (5) panel).

The lymph node uptake and B-cell colocalization 60 minutes after a 200-μl oral dose of 160 μM of Compound 2/POPC DilC₁₈ (5)-DS labeled liposomes were evaluated. DAPI staining was used to show the general structure of the lymph node section. CD45R-FITC was used to show B-cell localization. Cy5 channel, which detects DilC₁₈ (5) fluorescence, showed POPC liposome penetration into the lymph node. Merging FITC and Cy5 channels showed colocalization of B-cells and POPC liposomes. Similar to DOPC and DSPC, POPC seemed to penetrate deep into the lymph node. Colocalization was seen in both the bright and dim fluorescent regions, suggesting colocalization with both mature and immature B-cells (see FIG. 2F, arrows in the CD45R-FITC/DilC₁₈ (5) panel).

B-cell co-localization was analyzed. The averaged mean fluorescent intensity (FI) for B-cells in the analyzed sections was similar among all groups: 17.8 (5.9) (mean (SD)) for DMPC, 16.3 (8.7) for DOPC, 18.3 (7.8) for DSPC, and 25.4 (8.8) for POPC. Both uncorrected and corrected mean FI areas showed high variability for all formulations. Percent localization, however, showed less variability (Table 4) since it corrected for the number of B-cells in each section. Liposomes formulated with a saturated lipid (DSPC or DMPC) had better B-cell colocalization than those formulated with an unsaturated lipid (DOPC or POPC). The percent colocalization (mean (SD)) was 20.2% (3.9%) for DSPC and 10.3% (0.8%) for DMPC, whereas the percent colocalization was 6.8% (2.5%) for DOPC and 5.0% (1.6%) for POPC. Statistical analysis showed that DSPC liposomes were statistically better than the other three formulations (FIG. 2G).

TABLE 4
Percent B-Cell/Liposome Colocalization by Lipid Type

| | | | | Corrected | | | |
|---|---|---|---|---|---|---|---|
| | B-cells | | Mean FI of | mean FI for | | | |
| | Mean | RFI for each | colocalized | colocalized | % | | |
| | FI | formulation | area | area | Colocalization | Mean | SD |
| DMPC | 15.8 | 35.8 | 56.5 | 1.6 | 10.0 | 10.3 | 0.8 |
| | 24.4 | | 98.5 | 2.8 | 11.3 | | |
| | 13.2 | | 45.7 | 1.3 | 9.7 | | |
| DOPC | 22.4 | 73.2 | 76.5 | 1.0 | 4.7 | 6.8 | 2.5 |
| | 20.3 | | 89.5 | 1.2 | 6.0 | | |
| | 6.4 | | 44.6 | 0.6 | 9.6 | | |
| DSPC | 24.8 | 20.4 | 80.4 | 3.9 | 15.9 | 20.2 | 3.9 |
| | 20.6 | | 98.0 | 4.8 | 23.3 | | |
| | 9.6 | | 42.0 | 2.1 | 21.5 | | |
| POPC | 27.2 | 61.8 | 55.6 | 0.9 | 3.3 | 5.0 | 1.6 |
| | 33.2 | | 131.0 | 2.1 | 6.4 | | |
| | 15.8 | | 50.8 | 0.8 | 5.2 | | |

FITC-conjugated anti-TCRβ was observed in all samples, indicating the presence of T-cells. In most sections, T-cells were seen evenly distributed throughout. It was expected that T-cells would be localized in the paracortex of the lymph node. However, localized structures were observed in some, but not all, sections (FIG. 2H).

The lymph node uptake and T-cell colocalization 60 minutes after an oral dose of 200 μl of PBS containing 100 nM of DilC18 (5) were evaluated. DAPI staining shows the general structure of the lymph node section. TCRβ-FITC shows T-cell localization. Cy5 channel, which detects DilC₁₈ (5) fluorescence, showed no signal, indicating no dye uptake into the lymph node (FIG. 2I).

The lymph node uptake and T-cell colocalization 60 minutes after a 200-μl oral dose of 160 μM of Compound 2/DMPC DilC₁₈ (5)-DS labeled liposomes were evaluated. DAPI staining was used to show the general structure of the lymph node section. TCRβ-FITC was used to show T-cell localization. Cy5 channel, which detects DilC₁₈ (5) fluorescence, showed DMPC liposome penetration into the lymph node. Red fluorescence can be seen in clusters surrounding dark spots. Merging FITC and Cy5 channels showed colocalization of T-cells and DMPC liposomes (see FIG. 2J, arrows in the TCRβ-FITC/DilC₁₈ (5) panel).

The lymph node uptake and T-cell colocalization 60 minutes after a 200-μl oral dose of 160 μM of Compound 2/DOPC DilC₁₈ (5)-DS labeled liposomes were evaluated. DAPI staining was used to show the general structure of the lymph node section. TCRβ-FITC was used to show T-cell localization. Cy5 channel, which detects DilC₁₈ (5) fluorescence, showed DOPC liposome penetration into the lymph node. As in the DMPC treatment group, red fluorescence was observed in clusters surrounding dark spots. Merging FITC and Cy5 channels showed colocalization of T-cells and DOPC liposomes (see FIG. 2K, arrows in the TCRβ-FITC/DilC₁₈ (5) panel).

The lymph node uptake and T-cell colocalization 60 minutes after a 200-μl oral dose of 160 μM of Compound 2/DSPC DilC₁₈ (5)-DS labeled liposomes were evaluated. DAPI staining was used to show the general structure of the lymph node section. TCRβ-FITC was used to show T-cell localization. Cy5 channel, which detects DilC₁₈ (5) fluorescence, showed DSPC liposome penetration into the lymph node. As in the DOPC and DMPC treatment groups, red fluorescence was detected in circular clusters. However, red fluorescence was more generally distributed, throughout the lymph node section. Merging FITC and Cy5 channels showed colocalization of T-cells and DSPC liposomes (see FIG. 2L, arrows in the TCRβ-FITC/DilC₁₈ (5) panel).

The lymph node uptake and T-cell colocalization 60 minutes after a 200-μl oral dose of 160 μM of Compound 2/POPC DilC₁₈ (5)-DS labeled liposomes were evaluated. DAPI staining was used to show the general structure of the lymph node section. TCRβ-FITC was used to show T-cell localization. Cy5 channel, which detects DilC18 (5) fluorescence, showed POPC liposome penetration into the lymph node. As in the DSPC treatment group, red fluorescence was seen in circular clusters, as well as some more generally distributed throughout the lymph node section. Merging FITC and Cy5 channels showed colocalization of T-cells and DSPC liposomes (see FIG. 2M, arrows in the TCRβ-FITC/DilC₁₈ (5) panel).

T-cell co-localization was analyzed. The averaged mean fluorescent intensity (FI) for T-cells in the analyzed sections was similar among all groups, 13.7 (7.7) for DMPC, 11.8 (3.8) for DOPC, 8.1 (3.0) for DSPC, and 11.9 (3.3) (mean (SD)) for POPC. Both uncorrected and corrected mean FI areas showed higher variability than the variability observed in the sections used to evaluate B-cell colocalization regardless of formulation. Percent localization, however, showed substantially less variability for all formulation except DMPC (Table 5). Statistical analysis of the different groups showed that there was no difference among the groups. This is mainly driven by the variability in the DMPC group (global ANOVA reported in FIG. 2N). However, the pair-wise comparison showed that DSPC was statistically better than DOPC and POPC (FIG. 2N).

TABLE 5
Percent T-Cell/Liposome Colocalization by Lipid Type

| | | | | Corrected | | | |
|---|---|---|---|---|---|---|---|
| | T-cells | | Mean FI of | mean FI for | | | |
| | mean | | colocalized | colocalized | % | | |
| | FI | RFI | area | area | Colocalization | Mean | SD |
| DMPC | 5.1 | 35.8 | 104.0 | 2.9 | 57.1 | 26.9 | 26.2 |
| | 19.8 | | 81.6 | 2.3 | 11.5 | | |
| | 16.4 | | 70.1 | 2.0 | 12.0 | | |
| DOPC | 8.4 | 73.2 | 27.0 | 0.4 | 4.4 | 6.5 | 4.1 |
| | 15.8 | | 129.0 | 1.8 | 11.2 | | |
| | 11.2 | | 30.9 | 0.4 | 3.8 | | |
| DSPC | 8.0 | 20.4 | 44.0 | 2.2 | 27.1 | 23.6 | 3.7 |
| | 5.2 | | 21.0 | 1.0 | 19.8 | | |
| | 11.2 | | 54.9 | 2.7 | 24.0 | | |
| POPC | 8.8 | 61.8 | 51.6 | 0.8 | 9.5 | 7.6 | 1.8 |
| | 11.6 | | 54.0 | 0.9 | 7.5 | | |
| | 15.3 | | 55.4 | 0.9 | 5.9 | | |

Acyl chain length and saturation level affect the physical properties of the liposome. Lipids with high unsaturation or shorter chain tend to have lower melting temperatures and can create fluidic lipid membranes. In contrast, saturated lipids and those with longer acyl chains tend to have higher melting temperatures and may create solid liposomes with gel-like lipid membranes.

After oral administration, the liposome is expected to reach the absorption site in the intestine intact, be absorbed, and drain into the afferent lymph node where it undergoes immune sampling. The data support the notion that orally administered liposomes can reach the mesenteric lymph node. Furthermore, exposure to immune cells within the lymph node were improved by changing the acyl chain length and saturation level.

DMPC and DSPC both have saturated acyl chains, and both outperformed DOPC and POPC. Furthermore, DSPC with its 18-carbon acyl chain outperformed DMPC which has a 14-carbon acyl chain. DSPC has the highest melting temperature (Tₘ=55.6° C.) among all the lipids tested. Liposomes synthesized with DSPC are expected to be solid at body temperature. Even with the incorporation of Compound 2, a DSPC liposome is expected to have the highest melting temperature among all other liposomes tested in this study.

The downside of having a solid liposome is the difficulty in loading its payload. This could be seen from the lower fluorescence of both DMPC and DSPC liposomes compared to DOPC and POPC, despite being re-hydrated with the same buffer containing the same amount of DilC₁₈ (5). This is not expected to impact the loading of Compound 2. Compound 2 has a 16-carbon tail that mimics a lysophospholipid, and was designed to act as a lipid and incorporated into the film of the liposome before the hydration step.

These data demonstrate that it is possible to target the lymphatic system with orally administered liposomes. The data further suggest that selecting the proper acyl chain for the lipid carrier can help in targeting specific structures, such as immune cells within the lymph node.

DSPC liposomes had better B-cell colocalization than DMPC, DOPC, and POPC. For T-cell colocalization, DSPC performed better than DOPC and POPC, although it was not statistically different from DMPC.

### Example 4. In vitro Dose-Response Analysis of Compound 1 and Compound 2 in Murine T-Cells

Compound 1 and Compound 2 were each formulated with dimyristoylphosphatidylcholine (DMPC) at a 30:70 drug to lipid molar ratio. Liposomes were synthesized according to Example 2 in PBS.

Splenocytes from naïve C57BL/6 mice were stained with CFSE, prepared, cultured, and dosed. Cells were seeded at 2×10⁵ cells/well. Cells were dosed with each compound from 48 μM to 1.5 μM at a log 2 dilution scheme. Cells were incubated for 72 hours before phenotyping for FoxP3⁺/TIM3⁺ CD4 T-cells. Flow cytometer analysis was performed to assess changes in percent FoxP3⁺/TIM3⁺ CD4 T-cells as a function of treatment and dose. Concentration versus response was fitted to a four- or five-parameter log-logistic model (using the “drc” package in “R”). The EC₅₀ and EC₉₀ values were obtained by model fitting.

Cells were observed via a microscope at the end of the incubation period. Cells dosed with 48 μM Compound 1 did not look healthy and were excluded from further analysis. All other cells looked healthy and suitable for flow cytometry analysis.

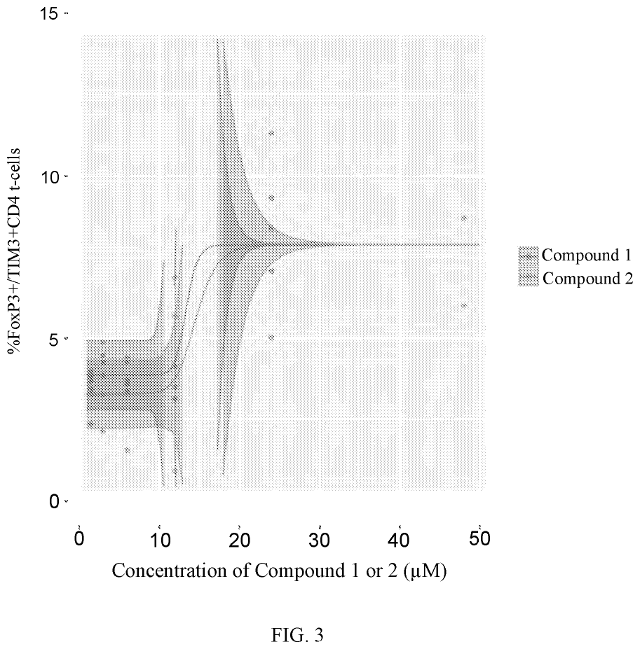

FoxP3⁺/TIM3⁺ double positive CD4 T-cells increased from a mean value of 3.65% (SD=0.2) and 3.21% (SD=1.47) for low dose Compound 1 and Compound 2, respectively, to 8.26% (SD=1.2) and 7.36% (SD=1.9) for high dose Compound 1 and Compound 2, respectively. Visual inspection of the model fit suggests that a 4-parameter dose-response model was sufficient to capture the data from Compound 2. However, due to the lack of plateau for Compound 1, the model was not able to give a reliable estimate of Eₘₐₓ and EC₅₀. To improve model fit, Eₘₐₓ was fixed to 7.9% which is the mean response for Compound 1 and Compound 2 at 10 μM, and 10 and 30 μM respectively. FIG. 3A shows the resulting model fit of the data to a 4-parameter dose-response model. Table 6 summarizes EC₅₀ and EC₉₀ values obtained by model fitting.

TABLE 6
Summary of Model Obtained EC₅₀ and
EC₉₀ Values by Treatment.

| Treatment | EC₅₀ mean (SE) | EC₉₀ mean (SE) |
|---|---|---|
| Compound 1 | 13.4 (22) | 15.0 (51) |
| Compound 2 | 14.6 (5.8) | 17.8 (13) |

Both Compound 1 and Compound 2 resulted in a dose-dependent increase in FoxP3⁺/TIM3⁺CD4 T-cells. Treatment with 10 μM Compound 1 resulted in a 126% increase in FoxP3⁺/TIM3⁺CD4 T-cells over low dose Compound 1. Similarly, treatment with 30 μM Compound 2 resulted in a 129% increase in FoxP3⁺/TIM3⁺CD4 T-cells over low dose Compound 2.

Compound 2 is expected to have more stability than Compound 1 since the ester linkage present in Compound 1 was replaced in Compound 2. Furthermore, Compound 2 showed less toxicity in vitro compared to Compound 1 at a 30 UM dose. Both Compound 1 and Compound 2 are attractive candidates for further development, however, Compound 2 may have some advantages from a stability and formulation standpoint.

### Example 5. Increase of FoxP3⁺/CD4⁺ T-cells by Compound 2 in Mice

Without being bound by theory, the engagement of Compound 2 with the TIM family of receptors is hypothesized to induce a tolerogenic immune response, e.g., increase tolerogenic FoxP3⁺/CD4⁺ T-cells (T-reg). Here, the pharmacodynamic (PD) effects of a single PO dose of Compound 2 in mice were evaluated after 5 days of dosing.

All animal studies were conducted under IACUC number B2020-91 in compliance with Tufts University/Tufts Medical Center & Human Nutrition Research Center on Aging. Animals (The Jackson Laboratory, Bar Harbor, ME, B57BL6) were housed four per cage and had access to food and water ad libitum.

Compound 2 was dissolved in DMSO and formulated with 1,2-dimyristoyl-sn-glycero-3-phosphocholine (DMPC) at a molar ratio of 10:90 Compound 2:DMPC, as described in Example 2. Animals were dosed according to Table 7. The doses were selected based on preliminary data suggesting 42 μM as the maximum efficacious dose in vitro.

TABLE 7
Dosing Groups

| | Dose μM/mouse | Volume μl | n |
|---|---|---|---|
| Compound | 4.2 (PO) | 100 | 3 |
| 2 | 14 (PO) | 100 | 3 |
| | 42 (PO) | 100 | 3 |
| | 140 (PO) | 100 | 3 |

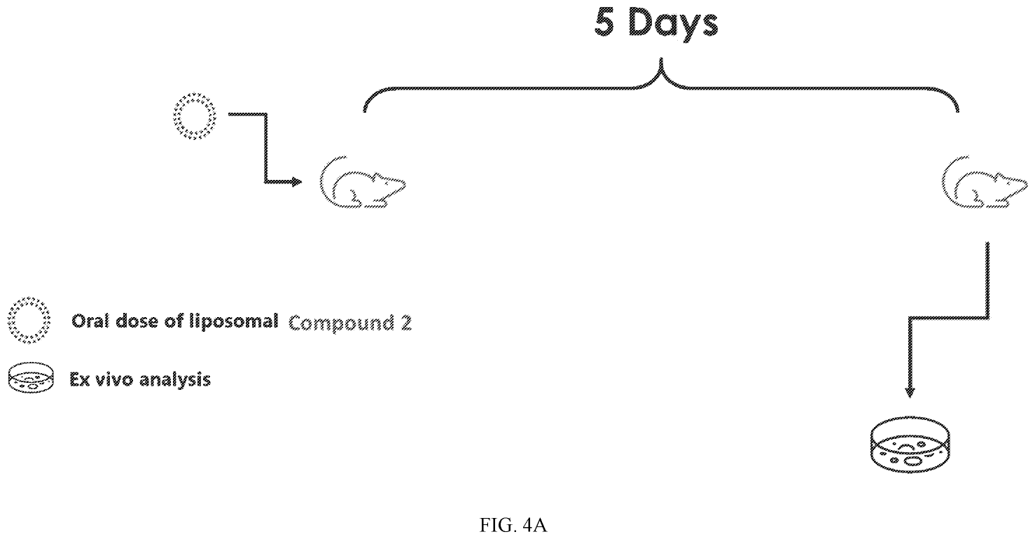

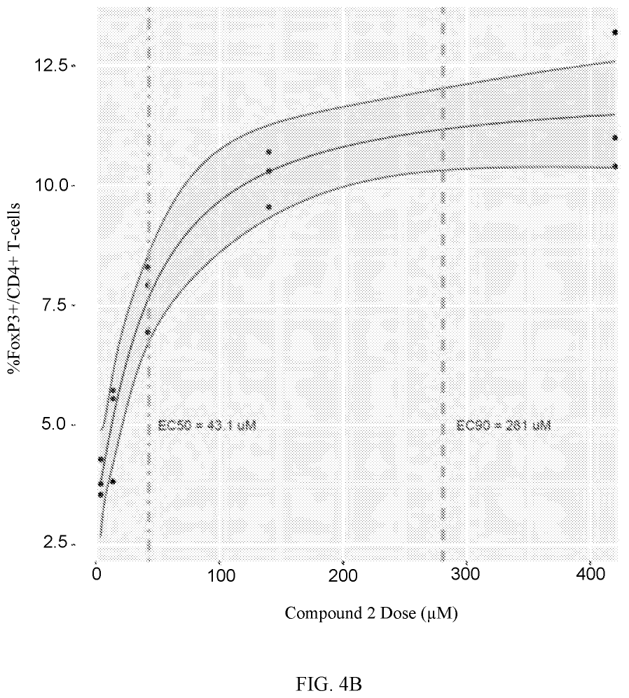

To evaluate the PD effects of a single dose of Compound 2 in mice, the animals were administered a single oral gavage as per their group assignment (Table 7). Five days after the oral dose, the animals were sacrificed and their spleens were collected for T-cell phenotyping. The study included an ex vivo analysis followed by a dose-response analysis (FIG. 4A). The spleens collected for splenocyte analysis were prepared into a single-cell suspension for cellular phenotyping by flow cytometry (eBioscience, Inc., San Diego, CA). Cells were stained and gated for CD4. Percent FoxP3⁺/CD4⁺ T-cells were evaluated using flow cytometry. A dose versus response curve was fitted to a four- or five-parameter log-logistic model (using the “drc” package in “R”). The EC₅₀ and EC₉₀ values were obtained by model fitting.

The percentage of the FoxP3⁺/CD4⁺ T-cells increased in a dose-dependent manner, from a mean value of 3.87% (SD=0.37) in the low dose group to 11.53% (SD=1.47) in the high dose group. The data were fitted to a four-parameter dose-response model. Table 8 shows the model estimated pharmacodynamic parameters and the associated standard errors. Based on this analysis, ED₅₀ is estimated at 43.1 μM, and ED₉₀ is estimated at 218 μM, after a single oral dose of Compound 2 (FIG. 4B).

TABLE 8
Model Estimated Pharmacodynamic Parameters

| Parameter | Mean | SD |
|---|---|---|
| E₀ | 3.3 | 1.1 |
| Eₘₐₓ | 12.1 | 1.2 |
| ED₅₀ μM | 43.1 | 13.4 |
| ED₉₀ μM | 281.0 | 262.0 |

Data from this study support the hypothesis that oral administration of Compound 2 can induce a tolerogenic immune response in a dose-dependent manner. Compared to the low dose group, a 219% increase of FoxP3⁺/CD4⁺ T-cells (T-regs) was observed in the high dose group. An increase above 100% (e.g., a doubling of FoxP3⁺/CD4⁺ T-cells) typically results in tolerance toward a target antigen. Thus, it is believed that repeat dosing at ED₅₀ would be sufficient for tolerance induction.

### Example 6. Prophylactic and Therapeutic Effects of Compound 2 in MOG₃₅₋₅₅-Induced Murine EAE Model

Multiple sclerosis is a chronic, often disabling, disease of the human central nervous system (CNS). Loss of tolerance to the myelin sheath causes the immune system to attack it and results in the clinical manifestation of the disease. This is mediated by pathogenic auto-reactive T-cells recognizing self-antigenic peptides in complex with major histocompatibility complex (MHC) molecules. There is no known cure for MS, however, blocking the ability of auto-reactive T-cells from entering the CNS proved to be an effective treatment option to ameliorate MS symptoms. Anti-α4 mAbs, such as Tysabri, have been approved for the management of MS. Other treatment options rely on generalized immune suppressive agents such as steroids.

Addressing the presence of auto-reactive T-cells and inducing a shift to higher tolerogenic T-cells may provide potentially curative therapy to MS. Compound 2 is a proposed T-cell immunoglobulin mucin protein family of receptors (TIM) agonist that can induce tolerogenic T-cells. TIM plays a key role in adaptive and innate immune response and has been associated with the regulation of autoimmunity and cancer. Several ligands are known to bind to TIM, including the phospholipid phosphatidylserine (PS).

The affinity of PS to different members in the TIM family varies substantially, with TIM3 having a lower affinity to PS than TIM4. However, all anti-TIM3 antibodies that have shown any functional efficacy in vivo and in vitro interfere with TIM3 binding to PS, suggesting that PS-TIM3 interaction is key in the TIM3 function, even at lower affinity.

The tolerogenic potential of PS has been exploited by tumors. For example, PS in the human ovarian tumor microenvironment can induce T cell signaling arrest. Furthermore, the PS-mediated T cell arrest was blocked with anti-PS antibodies. Collectively, published data on TIM and its role in immune tolerance suggest TIM as a potential target for the treatment of auto-immune disorders.

This study evaluates the effects of Compound 2, a TIM agonist, on disease onset, disease progression, and overall survival of mice in an experimental autoimmune (allergic) encephalomyelitis (EAE). EAE is considered to be the best model of multiple sclerosis (MS).

All animal studies are conducted under IACUC number B2020-91 and in compliance with Tufts University/Tufts Medical Center & Human Nutrition Research Center on Aging. Animals are housed at designated facilities within Tufts University-Tufts Medical Center & Human Nutrition Research Center on Aging. Animals are housed four in a cage and have access to food and water ad libitum.

Materials and reagents are provided in Table 9.

TABLE 9
Materials and Reagents

| | |
|---|---|
| Mice B57BL6 | CRL |
| MOG₃₅₋₅₅ | R&D |
| Complete Freund's | Thermo Fisher Scientific, |
| Adjuvant (CFA) | Waltham, MA |
| Pertussis Toxin (PT) | |

Compound 2 was formulated with 1,2-distearoyl-sn-glycero-3-phosphocholine (DSPC) at a molar ratio of 3.75:96.25 Compound 2:DSPC. Two test articles were formulated and tested. The first formulation was made with Compound 2:DSPC with MOG₃₅₋₅₅, referred to as Compound 2/MOG₃₅₋₅₅. Another formulation was made without MOG₃₃₋₅₅, and is referred to as Compound 2. DSPC was chosen based on superior mesenteric lymph node targeting and superior immune cell co-localization after oral administration. The ratio of Compound 2 to DSPC was chosen based on in vitro dose-response studies. Liposomes were prepared as described in Example 2, in PBS and extruded through a 100-micron filter. Animals were dose with 100 μl of 42 μM Compound 2 or 100 μl of 42 μM/1 μg Compound 2/MOG₃₅₋₅₅ per mouse PO QD. This dose was selected based on in vitro dose-response analysis.

Experimental autoimmune (allergic) encephalomyelitis (EAE) is considered the best non-clinical model of multiple sclerosis (MS). EAE is characterized by immune responses against CNS tissue and can be induced in animals by immunizing them against proteins of the CNS.

In the active EAE model, mice are immunized with MOG₃₅₋₅₅ peptide emulsified in Complete Freund's Adjuvant (CFA) by subcutaneous injection at the tail base (0.1 mL of emulsion/mouse) under anesthesia. On the day of injection (Day 0) and 2 days later, mice receive an intraperitoneal injection of Pertussis Toxin (PT) in PBS at 600 ng/mouse/dose (0.1 mL).

Symptoms typically develop in mice 9-14 days after immunization (Day 0). Daily observation and scoring of mice start on Day 9 and continue until the end of the study. Table 10 details the expected clinical symptoms and the scoring criteria used in this study.

TABLE 10
Mouse EAE Clinical Symptoms and Scoring Criteria

| Score | Clinical observations |
|---|---|
| 0.0 | No obvious changes in motor function compared to non-immunized mice: When |
| | picked up by the base of the tail, the tail has tension and is erect. Hind legs are usually |
| | spread apart. When the mouse is walking, there is no gait or head tilting. |
| 0.5 | Tip of the tail is limp: When picked up by the base of the tail, the tail has tension |
| | except for the tip. Muscle straining is felt in the tail, while the tail continues to move. |
| 1.0 | Limp tail: When picked up by the base of the tail, instead of being erect, the whole tail |
| | drapes over one's finger. Hind legs are usually spread apart. No signs of tail movement |
| | are observed. |
| 1.5 | Limp tail and hind leg inhibition: When picked up by the base of the tail, the whole |
| | tail drapes over one's finger. When the mouse is dropped on a wire rack, at least one |
| | hind leg falls through consistently. Walking is very slightly wobbly. |
| 2.0 | Limp tail and weakness of hind legs: a) When picked up by the base of the tail, the |
| | legs are not spread apart but held closely together. When the mouse is observed |
| | walking, it has a clearly apparent wobbly walk. One foot may have toes dragging, but |
| | the other leg has no apparent inhibitions of movement. Or, b) Mouse appears to be at a |
| | score 0.0, but there are obvious signs of head tilting when the walk is observed. The |
| | balance is poor. |
| 2.5 | Limp tail and dragging of hind legs: a) Both hind legs have some movement, but both |
| | are dragging at the feet (mouse trips on hind feet). Or b) No movement in one |
| | leg/completely dragging one leg, but movement in the other leg. Or c) EAE severity |
| | appears mild when picked up (as score 0.0-1.5), but there is a strong head tilt that |
| | causes the mouse to occasionally fall over. |
| 3.0 | a) Limp tail and complete paralysis of hind legs (most common). Or b) Limp tail and |
| | almost complete paralysis of hind legs. One or both hind legs are able to paddle, but |
| | neither hind leg is able to move forward of the hind hip. Or c) Limp tail with paralysis |
| | of one front and one hind leg. Or d) ALL of the severe head tilting, |
| | Walking only along the edges of the cage, |
| | Pushing against the cage wall, |
| | Spinning when picked up by the base of the tail. |
| 3.5 | Limp tail and complete paralysis of hind legs. In addition: The mouse is moving |
| | around the cage, but when placed on its side, is unable to right itself. Hind legs are |
| | together on one side of the body. Or b) Mouse is moving around the cage, but the |
| | hindquarters are flat like a pancake, giving the appearance of a hump in the front |
| | quarters of the mouse. |
| 4.0 | Limp tail, complete hind leg, and partial front leg paralysis: Mouse is minimally |
| | moving around the cage but appears alert and feeding. |
| | Often euthanasia is recommended after the mouse scores 4.0 for 2 days. However, with |
| | daily s.c. fluids most C57BL/6 mice may recover to 3.5 or 3.0, while SJL mice may |
| | fully recover even if they reach a score of 4.0 at the peak of disease. When the mouse |
| | is euthanized because of severe paralysis, a score of 5.0 is entered for that mouse for |
| | the rest of the experiment. |
| 4.5 | Complete hind and partial front leg paralysis, no movement around the cage. The |
| | mouse is not alert. |
| | The mouse has minimal movement in the front legs. The mouse barely responds to |
| | contact. |
| | Euthanasia is recommended. When the mouse is euthanized because of severe |
| | paralysis, a score of 5.0 is entered for that mouse for the rest of the experiment. |
| 5.0 | The mouse is spontaneously rolling in the cage (euthanasia is recommended). |

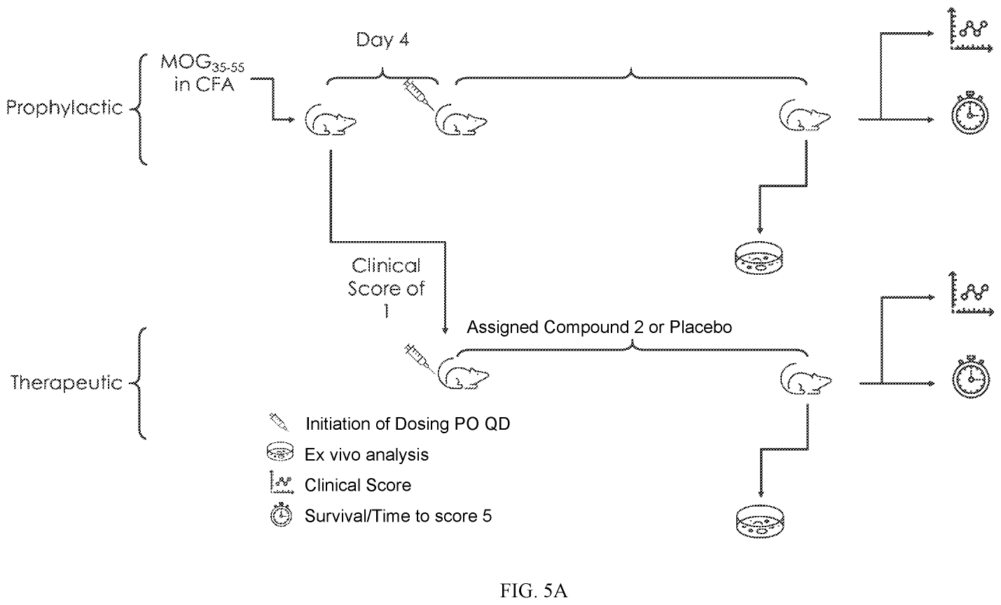

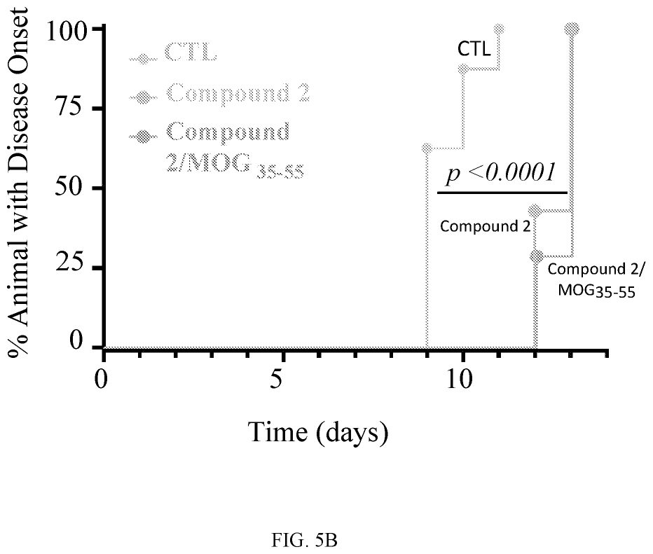

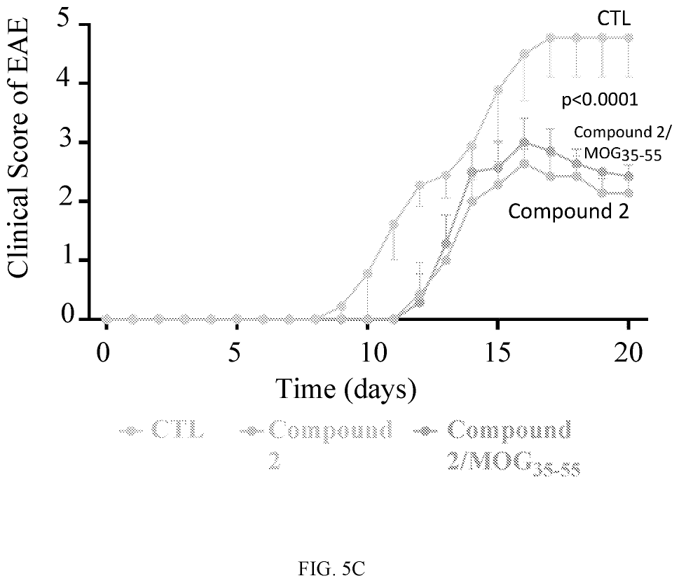

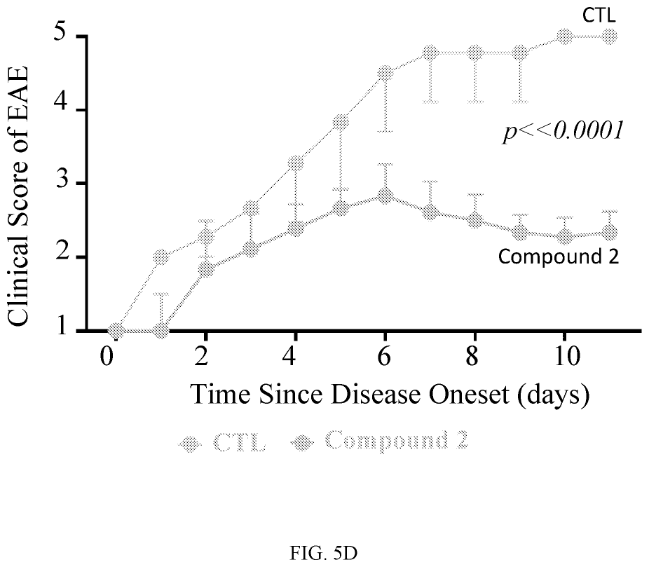

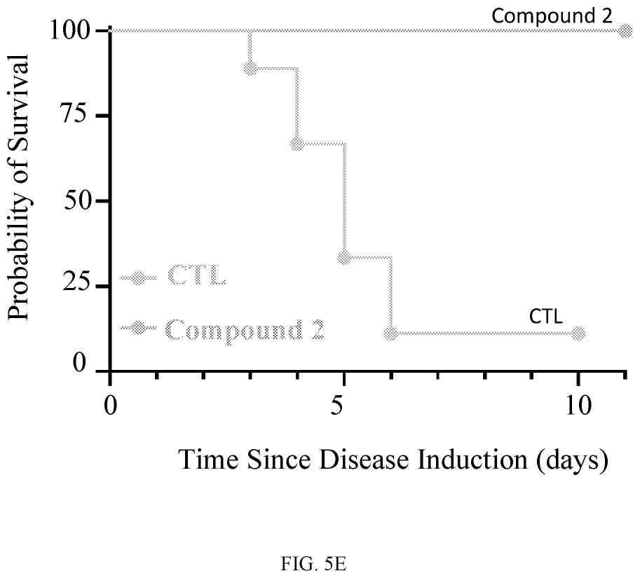

This study was designed to evaluate the prophylactic and therapeutic effects of Compound 2 in the EAE mouse model, and to evaluate the effects of antigen co-formulation with Compound 2. In the context of auto-immune diseases, it is hypothesized that co-formulation/co-presentation of antigen is not required since the antigen is expressed endogenously and auto-reactive T-cells are abundant due to the disease. FIG. 5A shows an outline of the study.

As shown in FIG. 5A, treatment in the prophylactic groups started four days after the mice were immunized with MOG₃₅₋₅₅. Mice were dosed with 100 μl of 42 μM Compound 2 or 100 μl of 42 μM of Compound 2 co-formulated with 1 μg of MOG₃₅₋₅₅ (Compound 2/MOG₃₅₋₅₅) orally QD for the duration of the study.

To evaluate the therapeutic effects of Compound 2, treatment was initiated after the first clinical symptoms were observed. Once a mouse had a clinical score of 1 as per Table 11, it was assigned to the treatment arm or the control arm. The treatment arm received 100 μl of 42 μM Compound 2 PO QD for the remainder of the study. Mice in all groups were monitored and scored daily for clinical symptoms. Once a mouse reached a score of five as per Table 11 or was found dead in the cage it was marked as an event in the survival analysis.

At the end of the study, spleens were collected from a representative sample of mice from each group for splenocyte analysis. A single-cell suspension was prepared for cellular phenotyping by flow cytometry. Cells were stained and gated for CD4⁺T-cells. Percent FoxP3⁺/CD4⁺ T-cells were evaluated by flow cytometry.

To evaluate the prophylactic effects of Compound 2 and Compound 2/MOG₃₅₋₅₅, in the EAE model, disease onset was evaluated. Disease onset was delayed in both Compound 2 and Compound 2/MOG₃₅₋₅₅ treatment groups compared to the control group. Disease onset in the control group started on day 9 after immunization versus day 12 in both treatment groups. All animals in the treatment groups showed symptoms by day 13 versus 11 in the control group. Time to event statistical analysis indicates that this was a statistically significant delay. There was no difference between Compound 2 and Compound 2/MOG₃₅₋₅₅ (FIG. 5B).

After disease onset, clinical scores progressed as expected in all arms. However, animals treated with Compound 2 and Compound 2/MOG₃₅₋₅₅ had less severe symptoms compared to the control group. Peak symptoms in the control group were observed on day 17 with a mean score of 4.77 (SD=0.67) versus 2.6 (SD=0.23) and 2.9 (SD=0.41) for the Compound 2 and Compound 2/MOG₃₅₋₅₅ treated groups respectively. A recovery phase was not observed in the control group since most animals in the control group died of the disease (FIG. 5C). However, in both Compound 2 and Compound 2/MOG₃₅₋₅₅ treatment groups, symptoms began to subside, and by end of the study, the mean scores were 2.1 (SD=0.23) and 2.4 (SD=0.23) for the Compound 2 and Compound 2/MOG₃₅₋₅₅, respectively (FIG. 5C).

After disease onset, the animals treated with Compound 2 had a one-day delay in disease progression followed by milder symptoms throughout the remainder of the study compared to untreated animals (FIG. 5D). Peak symptoms in the control group were observed on day 7 after the onset of disease with a mean score of 4.77 (SD=0.67) versus 2.8 (SD=0.43) on day 6 after the onset of symptoms in the treatment group. Symptoms in the treatment group subsided after the peak reaching a mean of 2.33 (SD=0.29) by day 10 after the onset of symptoms. Remission was not observed in the control group due to the high mortality in the untreated group.

A score of five or spontaneous death of any mouse was defined as an event for the survival analysis. The mortality rate in the untreated group was high in this study, with the first mouse dying three days after disease onset. Overall, eight out of nine animals spontaneously died or were euthanized as per Table 11, versus zero out of nine in the Compound 2 treated group (FIG. 5E). This was an unusually aggressive model, as typical mortality in EAE murine model is reported as less than 30%.

Flow cytometry analysis of splenocytes isolated from the last surviving mouse in the control group and randomly selected mice from each treatment group showed an increase in the Foxp3⁺/CD4⁺ T-cell in all treated mice compared to control. In the prophylactic group, the percent of Foxp3⁺/CD4⁺ T-cells were 10.4% and 7.02% for Compound 2 and Compound 2/MOG₃₅₋₅₅ treated mice, respectively, versus 3.20% for the control mice, representing a 225% and 119% increase over control, respectively. Similarly, the percent Foxp3⁺/CD4⁺ T-cell in the mouse treated with Compound 2 after the onset of symptoms was 26.1%, a 715% increase over the surviving mouse in the untreated group.

In this study, an aggressive form of the EAE model was induced in mice. This is evident from the high clinical score and mortality rate in the untreated group with eight out of nine animals reaching a score of 5. In contrast, the average clinical score in the control/untreated animals from a sample of EAE publications ranges from 3 to 4. Despite the aggressive form of EAE, prophylactic treatment with oral Compound 2 and Compound 2/MOG₃₅₋₅₅ delayed disease onset by at least 3 days, which is on par with the observation made with anti-α4 antibody treatment in an EAE model as reported by Kent et al. There was no difference between Compound 2 and Compound 2/MOG₃₅₋₅₅, suggesting that co-administration/co-presentation of antigen is not needed in autoimmune models.

Animals treated with Compound 2 after the onset of symptoms had better survival and lower clinical scores than those in the untreated group. By the end of the study, animals treated with Compound 2 had a mean clinical score that was 54% lower than the mean clinical score in the untreated arm. In comparison, Kent et al. reported a 66% decrease in the mean clinical score at the peak of response in the anti-α4 treated mice (day 15 of the study) compared to the mean clinical score in the untreated mice.

The ex vivo analysis of splenocytes isolated from representative animals from each group supports the proposed mechanism of action of Compound 2—an increase in tolerogenic T-cells was observed in all treated groups versus untreated animals.

### Example 7. Liposome Formulation of Compound 2 with DMPC, GL67

To test dissolution of Compound 2 in DMPC and GL67 (N4-cholesteryl-spermine HCl salt), Compound 2 (3 mg) was incubated in DMPC (42.47 mg) and GL67 (2.669 mg) at room temperature (RT) in 3 ml chloroform solution at a molar ratio of Compound 2:DMPC:GL67 of 10:85:5. Specifically, 3.0 mg Compound 2 were weighed and mixed with 3 ml chloroform in volumetric flask. The flask was sonicated for five minutes, then briefly vortexed, and incubated at RT overnight. After the overnight incubation, the flask was sonicated for five minutes, then briefly vortexed. 42.47 mg DMPC were added to the flasks. A clear solution with no cloudiness was observed. 2.669 mg GL67 were added to the flask, and the flask was sonicated for one to three minutes. A clear solution was observed following sonication.

The clear solution was transferred into a 5-ml, round-bottomed flask and loaded it on the rotavapor. The rotavapor was initially run at RT at a vacuum pressure of 800 mbar and a speed of 5, then the temperature of the water bath was increased to 37° C. The vacuum pressure was then decreased to 600 mbar for 15 minutes, then further decreased to 400 mbar for 30 minutes. After approximately one hour on the rotavapor, the organic solvent was completely evaporated, and a thin film had formed on the round-bottomed flask.

Next, hydration buffer was prepared for rehydration of the thin film. Specifically, 880 mg hydroxypropyl beta cyclodextrin (HPBCD) were weighed, and dissolved in normal saline in a 50-ml conical tube (Q.S. to 11 ml). Then AAV9-CMV^{Chry} (3×10⁶, from Vigene Biosciences, a Charles River company) was added to the hydration buffer.

The hydration buffer was then used to rehydrate the film. This was done by adding the buffer to the round-bottomed flask and attaching the round-bottomed flask to the rotavapor under vacuum. The gentle rotation of the rotavapor resulted in rehydration of the thin film and formation of liposomes. AAV9 particles became trapped in the aqueous center of the newly formed liposomes as the thin film was being rehydrated into liposomes.

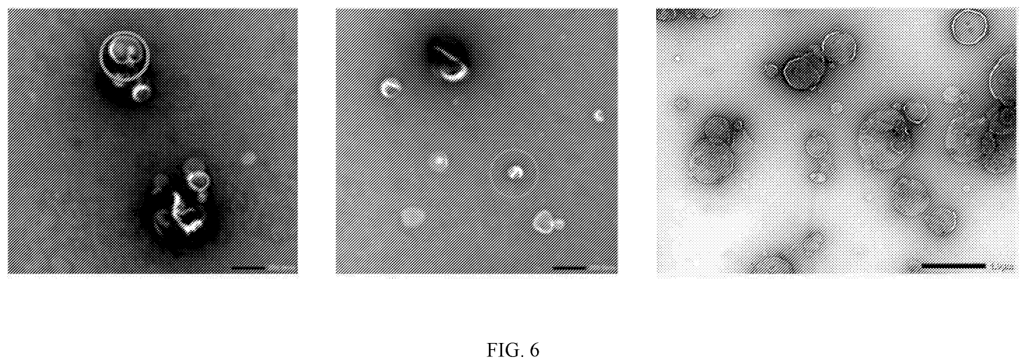

The resulting liposomes were imaged after rehydration using transmission electron microscopy (TEM). FIG. 6 shows representative TEM images of AAV9-CMV^{Chry} encapsulated in DMPC:GL67: Compound 2 (85:5:10) liposomes, where the red arrows and circles indicate encapsulated AAV9-CMV^{Chry}.

### Example 8. Increase of FoxP3⁺/CD4⁺ T-cells by Compound 2 in Mice

The PD effects of a single PO dose of DMPC:GL67: Compound 2 (85:5:10) liposomes encapsulating AAV9-GFP were evaluated in mice after 5 days of dosing. Liposomes were synthesized in accordance with the procedure described in Example 7, except that AAV9-CMV^{Chry} was replaced with AAV9-GFP (from Vigene Biosciences, a Charles River company). The experiment was conducted as described in Example 5 using the dosing groups described in Table 11.

TABLE 11
Dosing Groups

| | Liposomes | n |
|---|---|---|
| Naïve | — | 3 |
| 10⁶ AAV9-GFP Formulation | 100 μg | 3 |

In the naïve group, the percentages of FoxP3⁺/CD4⁺ T-cells measured by flow cytometry were 4.65, 3.91 and 5.01. In the group treated with the formulation of DMPC:GL67: Compound 2 (85:5:10) liposomes encapsulating 10⁶ AAV9-GFP, the percentages of FoxP3⁺/CD4⁺ T-cells measured by flow cytometry were 7.94, 9.37 and 8.15. Thus, oral administration of a single dose of 100 μg/mouse of 10⁶ AAV9-GFP encapsulated in DMPC:GL76: Compound 2 (85:5:10) liposomes enhanced T_{reg} expression five days after administration.

### REFERENCES

1. Wolf, Y., Anderson, A. C. & Kuchroo, V. K. TIM3 comes of age as an inhibitory receptor. Nat Rev Immunol 20, 173-185 (2020). https://doi.org/10.1038/s41577-019-0224-6
2. Freeman G J, Casasnovas J M, Umetsu D T, DeKruyff R H. TIM genes: a family of cell surface phosphatidylserine receptors that regulate innate and adaptive immunity. Immunol Rev. 2010; 235 (1): 172-189. doi: 10.1111/j.0105-2896.2010.00903.x.
3. Albacker L A, Karisola P, Chang Y J, et al. TIM-4, a receptor for phosphatidylserine, controls adaptive immunity by regulating the removal of antigen-specific T cells. J Immunol. 2010; 185 (11): 6839-6849. doi: 10.4049/jimmunol. 1001360
4. Santiago C, Ballesteros A, Martínez-Muñoz L, et al. Structures of T cell immunoglobulin mucin protein 4 show a metal-Ion-dependent ligand binding site where phosphatidylserine binds. Immunity. 2007; 27 (6): 941-951. doi: 10.1016/j.immuni.2007.11.008
5. Sabatos-Peyton, C. A. et al. Blockade of Tim-3 binding to phosphatidylserine and CEACAM1 is a shared feature of anti-Tim-3 antibodies that have functional efficacy. Oncoimmunology 7, e1385690 (2018).
6. Kelleher R J Jr, Balu-Iyer S, Loyall J, et al. Extracellular Vesicles Present in Human Ovarian Tumor Microenvironments Induce a Phosphatidylserine-Dependent Arrest in the T-cell Signaling Cascade. Cancer Immunol Res. 2015; 3 (11): 1269-1278. doi: 10.1158/2326-6066.CIR-15-0086.
7. Ramadan A, Lucca L E, Carrie N, Desbois S, Axisa P P, Hayder M, Bauer J, Liblau R S, Mars LT. In situ expansion of T cells that recognize distinct self-antigens sustains autoimmunity in the CNS. Brain. 2016 May; 139 (Pt 5): 1433-46. doi: 10.1093/brain/aww032. Epub 2016 Mar. 21. PMID: 27000832.
8. Kent, S. J., Karlik, S. J., Cannon, C., Hines, D. K., Yednock, T. A., Fritz, L. C., & Horner, H. C. (1995). A monoclonal antibody to α4 integrin suppresses and reverses active experimental allergic encephalomyelitis. Journal of neuroimmunology, 58 (1), 1-10.
9. Serre L, Girard M, Ramadan A, Menut P, Rouquié N, Lucca L E, Mahiddine K, Leobon B, Mars L T, Guerder S. Thymic-Specific Serine Protease Limits Central Tolerance and Exacerbates Experimental Autoimmune Encephalomyelitis. J Immunol. 2017 Dec. 1; 199 (11): 3748-3756. doi: 10.4049/jimmunol. 1700667. Epub 2017 Oct. 23. PMID: 29061767.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A compound of the following structural formula: 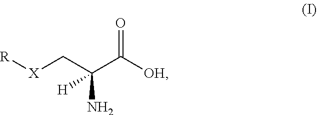
or a pharmaceutically acceptable salt thereof, wherein:
X is —N(R¹)C(O)—* or —N(R¹)C(O)O—*, wherein * indicates the point of attachment of X to R;
R is (C₅-C₃₀)alkyl or (C₅-C₃₀)alkenyl optionally substituted with one or more fluoro; and
R¹ is H or (C₁-C₅)alkyl,
provided the compound is not(S)-2-amino-3-((2E,4E)-hexa-2,4-dienamido) propanoic acid, (S)-2-amino-3-hexanamidopropanoic acid, (S)-2-amino-3-heptanamidopropanoic acid, (S)-2-amino-3-octanamidopropanoic acid or(S)-2-amino-3-palmitamidopropanoic acid, or a salt of any of the foregoing.

2. The compound of claim 1, wherein X is —N(R¹)C(O)—*

3. The compound of claim 1, wherein X is —N(R¹)C(O)O—*

4. The compound of any one of claims 1-3, wherein R is (C₅-C₃₀)alkyl optionally substituted with one or more fluoro.

5. The compound of any one of claims 1-3, wherein R is (C₁₅-C₂₀)alkyl or (C₁₅-C₂₀)alkenyl optionally substituted with one or more fluoro.

6. The compound of any one of claims 1-5, wherein R¹ is H.

7. The compound of claim 1, of the following structural formula: 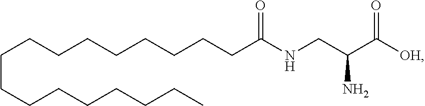 [SMILES: CCCCCCCCCCCCCCCCCC(=O)NC[C@H](N)C(=O)O]
or a pharmaceutically acceptable salt thereof.

8. The compound of claim 1, of the following structural formula: 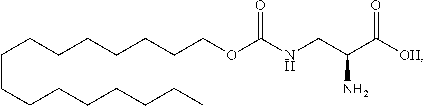 [SMILES: CCCCCCCCCCCCCCCCOC(=O)NC[C@H](N)C(=O)O]
or a pharmaceutically acceptable salt thereof.

9. A lipid particle comprising one or more lipids, or a pharmaceutically acceptable salt thereof, and (i) a compound of any one of claims 1-8, or a pharmaceutically acceptable salt thereof, or (ii) a compound of the following structural formula: 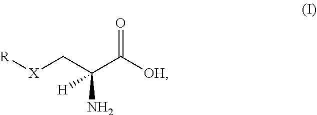
or a pharmaceutically acceptable salt thereof, wherein:
X is —N(R¹)C(O)—, —N(R¹)C(O)O—, —N(R¹)C(O)N(R²)—, —N(R¹)—, —N(R¹)SO₂—, —O—, —S—, —S(O)—, —S(O)₂— or —OP(O)₂O—;
R is (C₁-C₃₀)alkyl or (C₁-C₃₀)alkenyl optionally substituted with one or more fluoro;
R¹ is H or (C₁-C₅)alkyl; and
R² is H or (C₁-C₅)alkyl.

10. The lipid particle of claim 9, in the form of a liposome.

11. The lipid particle of claim 9 or 10, wherein the one or more lipids includes a phospholipid, or a pharmaceutically acceptable salt thereof.

12. The lipid particle of claim 11, wherein the phospholipid is a saturated phospholipid, or a pharmaceutically acceptable salt thereof.

13. The lipid particle of claim 12, wherein the phospholipid is dimyristoylphosphatidylcholine (DMPC) or 1,2-distearoyl-sn-glycero-3-phosphocholine 18:0 (DSPC).

14. The lipid particle of any one of claims 9-13, wherein the molar percentage of the compound of Structural Formula I, or a pharmaceutically acceptable salt thereof, in the lipid particle is from about 1% to about 50%.

15. The lipid particle of any one of claims 9-14, wherein the molar percentage of the one or more lipids, or a pharmaceutically acceptable salt thereof, in the lipid particle is from about 50% to about 99%.

16. The lipid particle of any one of claims 9-15, further comprising an antigen.

17. The lipid particle of claim 16, wherein the antigen is encapsulated within the lipid particle.

18. A composition comprising a plurality of lipid particles according to claim 16 or 17.

19. A composition comprising a plurality of lipid particles according to any one of claims 9-15.

20. The composition of claim 19, further comprising an antigen.

21. The composition of any one of claims 18-20, further comprising a pharmaceutically acceptable carrier.

22. A method of immunotolerizing a subject in need thereof to an antigen, comprising administering to the subject:
(i) the antigen, or an immunogenic fragment thereof, and a therapeutically effective amount of a composition of claim 19 or 21; or
(ii) a therapeutically effective amount of a composition of claim 18, 20 or 21, wherein the antigen in the composition is the antigen to which the subject is being immunotolerized, or an immunogenic fragment of the antigen to which the subject is being immunotolerized.

23. A method of inducing a population of regulatory T-cells in a subject, comprising administering to the subject a therapeutically effective amount of a composition of any one of claims 18-21.

24. A method of inducing a population of regulatory T-cells in a subject in response to an antigen, comprising administering to the subject a therapeutically effective amount of a composition of claim 18, 20 or 21, wherein the antigen in the composition is the antigen in response to which the population of regulatory T-cells is being induced, or an immunogenic fragment of the antigen in response to which the population of regulatory T-cells is being induced.

25. A method of inhibiting or reducing an antigen-specific antibody titer in a subject, comprising administering to the subject:
(i) the antigen and a therapeutically effective amount of a composition of claim 19 or 21; or
(ii) a therapeutically effective amount of a composition of claim 18, 20 or 21, wherein the antigen in the composition is the antigen for which the antibody titer is being inhibited or reduced, or an immunogenic fragment of the antigen for which the antibody titer is being inhibited or reduced.

26. A method of increasing the activity or level of tolerogenic T-cells in a subject, comprising administering to the subject a therapeutically effective amount of a composition of any one of claims 18-21.

27. A method of inducing a population of regulatory B-cells in a subject, comprising administering to the subject a therapeutically effective amount of a composition of any one of claims 18-21.

28. The method of claim 22, 24 or 25, wherein the antigen, or the immunogenic fragment thereof, and the composition are co-administered.

29. The lipid particle of claim 16 or 17, the composition of claim 18, 20 or 21, or the method of any one of claims 22, 24, 25 and 28, wherein the antigen is a foreign antigen.

30. The lipid particle, composition or method of claim 29, wherein the foreign antigen is an antigenic therapy.

31. The lipid particle, composition or method of claim 29 or 30, wherein the foreign antigen is a therapeutic protein.

32. The lipid particle, composition or method of any one of claims 29-31, wherein the foreign antigen is an enzyme replacement therapy.

33. The lipid particle, composition or method of claim 29 or 30, wherein the foreign antigen is a gene therapy.

34. A method of treating a disease, disorder or condition in a subject in need thereof with an antigenic therapy, comprising administering to the subject:
(i) a therapeutically effective amount of the antigenic therapy and, in an amount sufficient to immunotolerize the subject to the antigenic therapy, a composition of claim 19 or 21; or
(ii) a therapeutically effective amount of a composition of claim 18, 20 or 21, wherein the antigen in the composition is the antigenic therapy.

35. The method of claim 34, wherein the antigenic therapy and the composition are co-administered.

36. The method of claim 34 or 35, further comprising administering to the subject a therapeutically effective amount of the antigenic therapy in the absence of the composition.

37. The method of any one of claims 34-36, wherein the antigenic therapy is enzyme replacement therapy.

38. The method of any one of claims 34-36, wherein the antigenic therapy is gene therapy.

39. The lipid particle, composition or method of claim 33 or the method of claim 38, wherein the gene therapy comprises DNA, RNA or DNA and RNA, and a viral vector.

40. The lipid particle, composition or method of claim 39, wherein the viral vector is an adeno-associated virus (AAV).

41. The lipid particle, composition or method of claim 40, wherein the AAV is AAV9.

42. A method of treating an autoimmune disorder in a subject in need thereof, comprising administering to the subject:
(i) a therapeutically effective amount of a composition of claim 19 or 21; or
(ii) a therapeutically effective amount of a composition of claim 18, 20 or 21, wherein the antigen in the composition is a self-antigen associated with the autoimmune disorder, or an immunogenic fragment thereof.

43. The method of any one of claims 22-42, further comprising administering to the subject an additional therapeutic agent.

44. The method of any one of claims 22-43, wherein the composition is administered orally.
